(19) United States
(12) Patent Application Publication
(10) Pub. No.: US 2023/0416942 A1
(43) Pub. Date: Dec. 28, 2023

(54) Nanobody Compositions and Methods of Use of the Same

(71) Applicant: YALE UNIVERSITY, New Haven, CT (US)

(72) Inventors: Aaron Ring, New Haven, CT (US); Feimei Liu, New Haven, CT (US)

(21) Appl. No.: 18/042,385

(22) PCT Filed: Aug. 20, 2021
(86) PCT No.: PCT/US21/46858

§ 371 Date: Feb. 21, 2023

Related U.S. Application Data
(60) Provisional application No. 63/068,586, filed on Aug. 21, 2020.

(51) Int. Cl. C40B 30/04; G16B 40/00; C07K 16/00

(52) U.S. Cl. CPC C40B 30/04; G16B 40/00; C07K 2317/569; C07K 2317/24; C07K 16/005

(57) ABSTRACT

The invention provides methods for identifying nanobodies that bind with high affinity to secreted proteins or extracellular proteins, as well and compositions comprising the identified nanobodies and methods of use thereof.

### CROSS REFERENCE TO RELATED APPLICATIONS

This application claims priority to U.S. Provisional Application No. 63/068,586, filed Aug. 21, 2020 which is hereby incorporated by reference herein in its entirety.

### BACKGROUND OF THE INVENTION

Antibody discovery remains a labor and resource-intensive process involving immunization of animals or use of synthetic libraries with a molecular selection system such as phage display. Antibodies discovery campaigns thus typical focus on a single antigen target at one time. Currently, no solution exists that offers high-throughput selection of antibodies against many antigens simulatenously. Increases in throughput are typically accomplished with the use of robotics to perform multiple individual antibody selections in parallel. Accordingly, there exists a need for improved methods for the identification of antibodies, or antibody fragments, that bind to their target with high affinity and for novel antibodies, or antibody fragments, that can be used for the detection, diagnosis, prevention and treatment of diseases or disorders. The present invention meets this need.

### SUMMARY

In one embodiment, the invention relates to an isolated antibody or antibody fragment that specifically binds to a target protein or peptide, wherein the target protein or peptide is selected from the group consisting of a secreted protein or peptide and an extracellular protein or peptide.

In one embodiment, the antibody or antibody fragment is a monoclonal antibody, a polyclonal antibody, a single chain antibody, an immunoconjugate, a glycoengineered antibody, or a bispecific antibody or other multispecific antibody.

In one embodiment, the antibody is a single chain antibody. In one embodiment, the single chain antibody is a nanobody.

In one embodiment, the nanobody has a predicted binding score of at least 0.5. In one embodiment, the predicted binding score is determined using a machine learning algorithm wherein the machine learning algorithm comprises a multi-class classifier based on a logistic regression model.

In one embodiment, the antibody or antibody fragment specifically binds to the secreted or extracellular protein or peptide with an affinity of at least 10⁻⁶ M.

In one embodiment, the antibody or antibody fragment comprises a therapeutic agent or a detection moiety.

In one embodiment, the antibody is a humanized antibody, a chimeric antibody, a fully human antibody, or an antibody mimetic.

In one embodiment, the secreted or extracellular protein or peptide is selected from the group consisting of a target provided in Table 2.

In one embodiment, the extracellular protein or peptide is CCL16, CD302, CD3D, CD3E, CEACAM4, IL10RA, IL13, IL17A, IL1RN, IL21, PROCR, TIGIT, TNF, TNFRSF17, or TNFSF18.

In one embodiment, the antibody or antibody fragment comprises at least one of a) the heavy chain CDR1 sequence of SEQ ID NO:1-SEQ ID NO:42890; b) the heavy chain CDR2 sequence of SEQ ID NO:1-SEQ ID NO:42890; or c) the heavy chain CDR3 sequence of SEQ ID NO:1-SEQ ID NO:42890.

In one embodiment, the antibody or antibody fragment comprises each of: a) the heavy chain CDR1 sequence, b) the heavy chain CDR2 sequence and c) the heavy chain CDR3 sequence SEQ ID NO:1-SEQ ID NO:42890, wherein each of the CDR1, CDR2 and CDR3 sequence are from the same SEQ ID NO.

In one embodiment, the antibody or antibody fragment comprises an amino acid sequence as set forth in any one of SEQ ID NO:1-SEQ ID NO:42890, or a fragment thereof. In one embodiment, the fragment of SEQ ID NO:1-SEQ ID NO:42890 comprises the CDR1, CDR2 and CDR3 sequences of a nanobody as set forth in SEQ ID NO:1-SEQ ID NO:42890.

For each of the nanobodies as set forth in SEQ ID NO:1-SEQ ID NO:42890, the CDR1 sequence is defined as the region flanked by GSLRLSCAAS (SEQ ID NO:42891) . . . GWYRQAPGKE (SEQ ID NO:42892); the CDR2 sequence is defined as the region flanked by WYRQAPGKER (SEQ ID NO:42893) . . . ADSVKGRFTI (SEQ ID NO:42894); and CDR3 is defined as the region flanked by KPEDTAVYYC (SEQ ID NO:42895) . . . WGQGTQVTVS (SEQ ID NO:42896). For example, the CDR1, CDR2, and CDR3 of SEQ ID NO:1 are amino acid residues 26-34, amino acid residues 46-59, and amino acid residues 96-111, respectively.

In one embodiment, the invention relates to a composition comprising an isolated antibody or antibody fragment that specifically binds to a target protein or peptide, wherein the target protein or peptide is selected from the group consisting of a secreted protein or peptide and an extracellular protein or peptide.

In one embodiment, the invention relates to a nucleic acid molecule encoding an isolated antibody or antibody fragment that specifically binds to a target protein or peptide, wherein the target protein or peptide is selected from the group consisting of a secreted protein or peptide and an extracellular protein or peptide.

In one embodiment, the invention relates to a composition comprising a nucleic acid molecule encoding an isolated antibody or antibody fragment that specifically binds to a target protein or peptide, wherein the target protein or peptide is selected from the group consisting of a secreted protein or peptide and an extracellular protein or peptide.

In one embodiment, the invention relates to an expression vector comprising a nucleic acid molecule encoding an isolated antibody or antibody fragment that specifically binds to a target protein or peptide, wherein the target protein or peptide is selected from the group consisting of a secreted protein or peptide and an extracellular protein or peptide.

In one embodiment, the invention relates to an isolated host cell comprising a nucleic acid molecule encoding an isolated antibody or antibody fragment that specifically binds to a target protein or peptide, wherein the target protein or peptide is selected from the group consisting of a secreted protein or peptide and an extracellular protein or peptide.

In one embodiment, the invention relates to a method of treating or preventing a disease or disorder in a subject in need thereof, the method comprising the step of administering an isolated antibody or antibody fragment that specifically binds to a target protein or peptide, wherein the target protein or peptide is selected from the group consisting of a secreted protein or peptide and an extracellular protein or peptide.

In one embodiment, the invention relates to a method of treating or preventing a disease or disorder in a subject in need thereof, the method comprising the step of administering a composition comprising an isolated antibody or antibody fragment that specifically binds to a target protein or peptide, wherein the target protein or peptide is selected from the group consisting of a secreted protein or peptide and an extracellular protein or peptide.

In one embodiment, the invention relates to a method of treating or preventing a disease or disorder in a subject in need thereof, the method comprising the step of administering a nucleic acid molecule encoding an isolated antibody or antibody fragment that specifically binds to a target protein or peptide, wherein the target protein or peptide is selected from the group consisting of a secreted protein or peptide and an extracellular protein or peptide.

In one embodiment, the invention relates to a method of treating or preventing a disease or disorder in a subject in need thereof, the method comprising the step of administering a composition comprising a nucleic acid molecule encoding an isolated antibody or antibody fragment that specifically binds to a target protein or peptide, wherein the target protein or peptide is selected from the group consisting of a secreted protein or peptide and an extracellular protein or peptide.

In one embodiment, the invention relates to a method of detecting a target secreted or extracellular protein or peptide in a sample, the method comprising: a) contacting the sample with the antibody or antibody fragment that specifically binds to a target protein or peptide, wherein the target protein or peptide is selected from the group consisting of a secreted protein or peptide and an extracellular protein or peptide, and b) detecting binding of the antibody or antibody fragment to the target secreted or extracellular protein or peptide.

In one embodiment, the invention relates to a method of diagnosing a disease or disorder in a subject in need thereof, the method comprising: a) contacting a biological sample of the subject with the antibody or antibody fragment that specifically binds to a target protein or peptide, wherein the target protein or peptide is selected from the group consisting of a secreted protein or peptide and an extracellular protein or peptide, b) determining the level of the target secreted or extracellular protein or peptide in the biological sample of the subject, c) comparing the level of the target secreted or extracellular protein or peptide in the biological sample of the subject with a comparator control, and d) diagnosing the subject with a disease or disorder associated with a differential level of the target secreted or extracellular protein or peptide when the level of the target secreted or extracellular protein or peptide in the biological sample of the subject of subject is significantly different when compared with the level of the target secreted or extracellular protein or peptide in the comparator control. In one embodiment, the method further comprises a step of administering a treatment to the subject that was diagnosed as having a disease or disorder.

In one embodiment, the comparator control is at least one selected from the group consisting of: a positive control, a negative control, a historical control, a historical norm, or the level of a reference molecule in the biological sample.

In one embodiment, the invention relates to a method of selecting an antibody or antibody fragment that specifically binds to a target protein or peptide, wherein the target protein or peptide is selected from the group consisting of a secreted protein or peptide and an extracellular protein or peptide, the method comprising the steps of: a) contacting a support comprising a display library of target secreted and extracellular proteins or peptides with a phage display library of candidate antibodies or antibody fragments, such that a complex is formed between the candidate antibody and the target secreted and extracellular proteins or peptide; b) cleaving the complex from the support using a protease; c) isolating the nucleic acid molecule encoding the antibody or antibody fragment; and d) sequencing the nucleic acid molecule encoding the antibody or antibody fragment.

In one embodiment, the method further comprises predicting the binding score of the antibody or antibody fragment using a machine learning algorithm, wherein the machine learning algorithm comprises a multi-class classifier based on a logistic regression model.

### BRIEF DESCRIPTION OF THE DRAWINGS

The foregoing summary, as well as the following detailed description of preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts a schematic representation of the comprehensive nanobody discovery campaign on 3,088 human extracellular and secreted proteins.

FIG. 2 depicts a schematic representation of the HAPPY selection workflow.

FIG. 3 depicts a schematic representation of affinity selection of phage displayed nanobodies on yeast displayed antigens.

FIG. 4 depicts a schematic representation of phage elution from yeast surface by 3C protease reaction.

FIG. 5A and FIG. 5B depict representative results of flow cytometry analysis of polyclonal selection output phage binding on antigen displaying yeast. FIG. 5A depicts schematics illustrating flow cytometric measurements of phage displaying binder nanobodies (left) and non-binder nanobodies (right). FIG. 5B depicts flow cytometric measurements showing binding of phage output from the deconvolution selection to individual antigen-yeast strains. Values of background corrected mean fluorescence intensity (MFI) are listed in the table. Among the 96 wells, 45 (47%) demonstrate clear phage binding signal over background (MFI>50).

FIG. 6A through FIG. 6C depict the characterization of individual recombinant nanobody Fc fusion proteins. FIG. 6A depicts representative results of ELISA assays. FIG. 6B depicts representative results of Biolayer Interferometry assays. FIG. 6C depicts a representative list of nanobodies and human protein antigens that were characterized by ELISA.

FIG. 7A and FIG. 7B depict a prediction of Binders via Machine Learning Modeling. FIG. 7A depicts a schematic representation of the machine learning model that predicts Binding Score. FIG. 7B depicts representative performance results of the machine learning model, using three different random partitions of training and testing datasets.

FIG. 8 depicts a correlation between machine learning predicted binding score and the probability of binding evaluated with experimental validation.

### DETAILED DESCRIPTION OF THE INVENTION

Even though antibodies are the most widely used class of protein binding tool and therapeutic drug format, there are serious flaws in their reliability. It was shown that fewer than half of around 6,000 routinely used commercial antibodies actually recognized their targets specifically. This invention relates, in part, to the development of a novel high-throughput screening technique (referred to herein as “HAPPY”) that allows for discovery of nanobodies against extracellular and secreted proteins in humans. This invention relates, in part, to nanobodies that bind to their target with high affinity, and the use of the nanobodies of the invention to bind to, or inhibit, their target. In some embodiments, the invention relates, in part, to methods of diagnosing, treating or preventing diseases and disorders diagnosable, preventable and treatable using the nanobodies of the invention.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Generally, the nomenclature used herein and the laboratory procedures in cell culture, molecular genetics, organic chemistry, and nucleic acid chemistry and hybridization are those well-known and commonly employed in the art.

Standard techniques are used for nucleic acid and peptide synthesis. The techniques and procedures are generally performed according to conventional methods in the art and various general references (e.g., Sambrook and Russell, 2012, Molecular Cloning, A Laboratory Approach, Cold Spring Harbor Press, Cold Spring Harbor, NY, and Ausubel et al., 2012, Current Protocols in Molecular Biology, John Wiley & Sons, NY), which are provided throughout this document.

The nomenclature used herein and the laboratory procedures used in analytical chemistry and organic syntheses described below are those well-known and commonly employed in the art. Standard techniques or modifications thereof are used for chemical syntheses and chemical analyses.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, or ±10%, or ±5%, or ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected/homeostatic) respective characteristic. Characteristics which are normal or expected for one cell, tissue type, or subject, might be abnormal for a different cell or tissue type.

The term “analog” as used herein generally refers to compounds that are generally structurally similar to the compound of which they are an analog, or “parent” compound. Generally analogs will retain certain characteristics of the parent compound, e.g., a biological or pharmacological activity. An analog may lack other, less desirable characteristics, e.g., antigenicity, proteolytic instability, toxicity, and the like. An analog includes compounds in which a particular biological activity of the parent is reduced, while one or more distinct biological activities of the parent are unaffected in the “analog.” As applied to polypeptides, the term “analog” may have varying ranges of amino acid sequence identity to the parent compound, for example at least about 70%, more preferably at least about 80%-85% or about 86%-89%, and still more preferably at least about 90%, about 92%, about 94%, about 96%, about 98% or about 99% of the amino acids in a given amino acid sequence the parent or a selected portion or domain of the parent. As applied to polypeptides, the term “analog” generally refers to polypeptides which are comprised of a segment of about at least 3 amino acids that has substantial identity to at least a portion of a binding domain fusion protein. Analogs typically are at least 5 amino acids long, at least 20 amino acids long or longer, at least 50 amino acids long or longer, at least 100 amino acids long or longer, at least 150 amino acids long or longer, at least 200 amino acids long or longer, and more typically at least 250 amino acids long or longer. Some analogs may lack substantial biological activity but may still be employed for various uses, such as for raising antibodies to predetermined epitopes, as an immunological reagent to detect and/or purify reactive antibodies by affinity chromatography, or as a competitive or noncompetitive agonist, antagonist, or partial agonist of a binding domain fusion protein function.

The term “antibody,” as used herein, refers to an immunoglobulin molecule which is able to specifically bind to a specific epitope of a binding partner molecule. Antibodies can be intact immunoglobulins derived from natural sources, or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab, Fab′, F(ab)2 and F(ab′)2, as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to at least one portion of an intact antibody and refers to the antigenic determining variable regions of an intact antibody. Examples of antibody fragments include, but are not limited to, Fab, Fab′, F(ab′)2, and Fv fragments, linear antibodies, sdAb (either V_{L} or V_{H}), camelid V_{HH} domains, scFv antibodies, and multi-specific antibodies formed from antibody fragments. The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it was derived. Unless specified, as used herein an scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}.

An “antibody heavy chain,” as used herein, refers to the larger of the two types of polypeptide chains present in antibody molecules in their naturally occurring conformations, and which normally determines the class to which the antibody belongs.

An “antibody light chain,” as used herein, refers to the smaller of the two types of polypeptide chains present in antibody molecules in their naturally occurring conformations. Kappa (κ) and lambda (λ) light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

A “chimeric antibody” refers to a type of engineered antibody which contains a naturally-occurring variable region (light chain and heavy chains) derived from a donor antibody in association with light and heavy chain constant regions derived from an acceptor antibody.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see, e.g., 1989, Queen et al., Proc. Natl. Acad Sci USA, 86:10029-10032; 1991, Hodgson et al., Bio/Technology, 9:421). A suitable human acceptor antibody may be one selected from a conventional database, e.g., the KABAT database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanized antibodies (see for example EP-A-0239400 and EP-A-054951).

The term “donor antibody” refers to an antibody (monoclonal, and/or recombinant) which contributes the amino acid sequences of its variable regions, CDRs, or other functional fragments or analogs thereof to a first immunoglobulin partner, so as to provide the altered immunoglobulin coding region and resulting expressed altered antibody with the binding specificity and neutralizing activity characteristic of the donor antibody.

The term “acceptor antibody” refers to an antibody (monoclonal and/or recombinant) heterologous to the donor antibody, which contributes all (or any portion, but in some embodiments all) of the amino acid sequences encoding its heavy and/or light chain framework regions and/or its heavy and/or light chain constant regions to the first immunoglobulin partner. In certain embodiments a human antibody is the acceptor antibody.

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4th Ed., U.S. Department of Health and Human Services, National Institutes of Health (1987). There are three heavy chain and three light chain CDRs (or CDR regions) in the variable portion of an immunoglobulin. Thus, “CDRs” as used herein refers to all three heavy chain CDRs, or all three light chain CDRs (or both all heavy and all light chain CDRs, if appropriate). The structure and protein folding of the antibody may mean that other residues are considered part of the binding region and would be understood to be so by a skilled person. See for example Chothia et al., (1989) Conformations of immunoglobulin hypervariable regions; Nature 342, p 877-883.

The term “framework” or “framework sequence” refers to the remaining sequences of a variable region minus the CDRs. Because the exact definition of a CDR sequence may be determined by different systems, the meaning of a framework sequence is subject to correspondingly different interpretations. The six CDRs (CDR-L1, -L2, and -L3 of light chain and CDR-H1, -H2, and -H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. An FR represents one of the four sub-regions, and FRs represents two or more of the four sub-regions constituting a framework region.

As used herein, an “immunoassay” refers to any binding assay that uses an antibody capable of binding specifically to a target molecule to detect and quantify the target molecule.

By the term “specifically binds,” as used herein with respect to an antibody, is meant an antibody which recognizes a specific binding partner molecule, but does not substantially recognize or bind other molecules in a sample. For example, an antibody or nanobody that specifically binds to a binding partner molecule from one species may also bind to that binding partner molecule from one or more species. But, such cross-species reactivity does not itself alter the classification of an antibody as specific. In another example, an antibody or nanobody that specifically binds to binding partner molecule may also bind to different allelic forms of the binding partner molecule. However, such cross reactivity does not itself alter the classification of an antibody as specific.

In some instances, the terms “specific binding” or “specifically binding”, can be used in reference to the interaction of an antibody, a protein, or a peptide with a second binding partner molecule, to mean that the interaction is dependent upon the presence of a particular structure (e.g., an antigenic determinant or epitope) on the binding partner molecule; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody. In some instances, the terms “specific binding” and “specifically binding” refers to selective binding, wherein the antibody recognizes a sequence or conformational epitope important for the enhanced affinity of binding to the binding partner molecule.

The term “epitope” has its ordinary meaning of a site on binding partner molecule recognized by an antibody or a binding portion thereof or other binding molecule, such as, for example, an scFv. Epitopes may be molecules or segments of amino acids, including segments that represent a small portion of a whole protein or polypeptide. Epitopes may be conformational (i.e., discontinuous). That is, they may be formed from amino acids encoded by noncontiguous parts of a primary sequence that have been juxtaposed by protein folding.

The phrase “biological sample” as used herein, is intended to include any sample comprising a cell, a tissue, or a bodily fluid in which expression of a nucleic acid or polypeptide can be detected. Examples of such biological samples include but are not limited to blood, lymph, bone marrow, biopsies and smears. Samples that are liquid in nature are referred to herein as “bodily fluids.” Biological samples may be obtained from a patient by a variety of techniques including, for example, by scraping or swabbing an area or by using a needle to obtain bodily fluids. Methods for collecting various body samples are well known in the art.

As used herein, “conjugated” refers to covalent attachment of one molecule to a second molecule.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of a mRNA molecule also consists of the nucleotide residues of the mRNA molecule which are matched with an anti-codon region of a transfer RNA molecule during translation of the mRNA molecule or which encode a stop codon. The coding region may thus include nucleotide residues comprising codons for amino acid residues which are not present in the mature protein encoded by the mRNA molecule (e.g., amino acid residues in a protein export signal sequence).

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

As used herein, the term “derivative” includes a chemical modification of a polypeptide, polynucleotide, or other molecule. In the context of this invention, a “derivative polypeptide,” for example, one modified by glycosylation, pegylation, or any similar process, retains binding activity. For example, the term “derivative” of binding domain includes binding domain fusion proteins, variants, or fragments that have been chemically modified, as, for example, by addition of one or more polyethylene glycol molecules, sugars, phosphates, and/or other such molecules, where the molecule or molecules are not naturally attached to wild-type binding domain fusion proteins. A “derivative” of a polypeptide further includes those polypeptides that are “derived” from a reference polypeptide by having, for example, amino acid substitutions, deletions, or insertions relative to a reference polypeptide. Thus, a polypeptide may be “derived” from a wild-type polypeptide or from any other polypeptide. As used herein, a compound, including polypeptides, may also be “derived” from a particular source, for example from a particular organism, tissue type, or from a particular polypeptide, nucleic acid, or other compound that is present in a particular organism or a particular tissue type.

The term “DNA” as used herein is defined as deoxyribonucleic acid.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting there from. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

An “effective amount” or “therapeutically effective amount” of a compound is that amount of compound which is sufficient to provide a beneficial effect to the subject to which the compound is administered.

The term “epitope” as used herein refers to a protein determinant capable of binding to an antibody. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents.

The term “high affinity” for binding domain polypeptides described herein refers to a dissociation constant (Kd) of at least about 10⁻⁶M, preferably at least about 10⁻⁷M, more preferably at least about 10⁻⁸M or stronger, more preferably at least about 10⁻⁹M or stronger, more preferably at least about 10⁻¹⁰M or stronger, for example, up to 10⁻¹²M or stronger. However, “high affinity” binding can vary for other binding domain polypeptides.

The term “inhibit,” as used herein, means to suppress or block an activity or function, for example, about ten percent relative to a control value. Preferably, the activity is suppressed or blocked by 50% compared to a control value, more preferably by 75%, and even more preferably by 95%. “Inhibit,” as used herein, also means to reduce the level of a molecule, a reaction, an interaction, a gene, an mRNA, and/or a protein's expression, stability, function or activity by a measurable amount or to prevent entirely. Inhibitors are compounds that, e.g., bind to, partially or totally block activity, decrease, prevent, delay activation, inactivate, desensitize, or down regulate a protein, a gene, and an mRNA stability, expression, function and activity, e.g., antagonists.

The terms “modulator” and “modulation” of a molecule of interest, as used herein in its various forms, is intended to encompass antagonism, agonism, partial antagonism and/or partial agonism of an activity associated the protease of interest. In various embodiments, “modulators” may inhibit or stimulate protease expression or activity. Such modulators include small molecules agonists and antagonists of a protease molecule, antisense molecules, ribozymes, triplex molecules, and RNAi polynucleotides, and others.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

“Isolated” means altered or removed from the natural state. For example, a nucleic acid or a peptide naturally present in its normal context in a living animal is not “isolated,” but the same nucleic acid or peptide partially or completely separated from the coexisting materials of its natural context is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine. The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “conservative substitution,” when describing a polypeptide, refers to a change in the amino acid composition of the polypeptide that does not substantially alter the activity of the polypeptide, i.e., substitution of amino acids with other amino acids having similar properties. Conservative substitution tables providing functionally similar amino acids are well known in the art. The following six groups each contain amino acids that are generally understood to represent conservative substitutions for one another: (1) Alanine (A), Serine (S), Threonine (T); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); and (6) Phenylalanine (F), Tyrosine (Y), Tryptophan (W) (see also, Creighton, 1984, Proteins, W. H. Freeman and Company). In addition to the above-defined conservative substitutions, other modifications of amino acid residues can also result in “conservatively modified variants.” For example, one may regard all charged amino acids as substitutions for each other whether they are positive or negative. In addition, conservatively modified variants can also result from individual substitutions, deletions or additions which alter, add or delete a single amino acid or a small percentage of amino acids, for example, often less than 5%, in an encoded sequence. Further, a conservatively modified variant can be made from a recombinant polypeptide by substituting a codon for an amino acid employed by the native or wild-type gene with a different codon for the same amino acid.

The term “RNA” as used herein is defined as ribonucleic acid. The term “recombinant DNA” as used herein is defined as DNA produced by joining pieces of DNA from different sources.

The term “recombinant polypeptide” as used herein is defined as a polypeptide produced by using recombinant DNA methods.

The term “single chain antibody” is an antibody that contains an antigen binding site that is composed of a single polypeptide chain. One example of a single chain antibody is a single-chain variable fragment (scFv) antibody, which is a fusion protein that contains the variable regions of the heavy (VH) and light chains (VL) of a classical antibody connected by a short linker peptide of about ten to about 25 amino acids. A single-chain antibody can also be obtained by immunization of a camelid (e.g., a camel, llama or alpaca) or a cartilaginous fish (e.g., a shark), which make antibodies that are composed of only heavy chains. A monomeric variable domain of a heavy chain antibody binds antigen.

By “pharmaceutically acceptable” it is meant, for example, a carrier, diluent or excipient that is compatible with the other ingredients of the formulation and generally safe for administration to a recipient thereof. As used herein, “pharmaceutically acceptable carrier” includes any material, which when combined with the conjugate retains the conjugates' activity and is non-reactive with the subject's immune systems. Examples include, but are not limited to, any of the standard pharmaceutical carriers such as a phosphate buffered saline solution, water, emulsions such as oil/water emulsion, and various types of wetting agents. Other carriers may also include sterile solutions, tablets including coated tablets and capsules. Typically such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well-known conventional methods.

The term “a population of cells that comprise a library of surface-tethered extracellular capture agents” refers to a population of that cells that expresses (i.e., “displays”) a surface-tethered capture agent on their exterior surface and the amino acid sequence of the capture agent differs from cell to cell.

The terms “patient,” “subject,” “individual,” and the like are used interchangeably herein, and refer to any animal, preferably a mammal, and most preferably a human, having a complement system, including a human in need of therapy for, or susceptible to, a condition or its sequelae. Thus, the individual may include, for example, dogs, cats, pigs, cows, sheep, goats, horses, rats, monkeys, and mice and humans.

The phrase “percent (%) identity” refers to the percentage of sequence similarity found in a comparison of two or more amino acid sequences. Percent identity can be determined electronically using any suitable software. Likewise, “similarity” between two polypeptides (or one or more portions of either or both of them) is determined by comparing the amino acid sequence of one polypeptide to the amino acid sequence of a second polypeptide. Any suitable algorithm useful for such comparisons can be adapted for application in the context of the invention.

A “therapeutic” treatment is a treatment administered to a subject who exhibits signs of pathology, for the purpose of diminishing or eliminating those signs.

“Therapeutically effective amount” is an amount of a compound of the invention, that when administered to a patient, ameliorates a symptom of the disease. The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art having regard to his own knowledge and to this disclosure.

The terms “treat,” “treating,” and “treatment,” refer to therapeutic or preventative measures described herein. The methods of “treatment” employ administration to a subject, in need of such treatment, a composition of the present invention, for example, a subject afflicted a disease or disorder, or a subject who ultimately may acquire such a disease or disorder, in order to prevent, cure, delay, reduce the severity of, or ameliorate one or more symptoms of the disorder or recurring disorder, or in order to prolong the survival of a subject beyond that expected in the absence of such treatment.

“Variant” as the term is used herein, is a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

This invention is based, in part, on the development of a method of detecting proteins or peptides that bind with high affinity to a target protein such as a secreted protein or extracellular protein. In some embodiments, the invention provides a library of antibodies or nanobodies that bind with high affinity to secreted or extracellular proteins. In some embodiments, the invention relates to methods of using the proteins or peptides (e.g, antibodies or nanobodies) of the invention to bind to their target protein. In some embodiments, the invention relates to methods of using the proteins or peptides (e.g, antibodies or nanobodies) of the invention to treat or prevent diseases or disorder. In various embodiments, the invention is directed to compositions and methods for treating a disease or disorder in an individual by administering to a subject in need thereof at least one protein or peptide (e.g, antibody or nanobody) of the invention.

In certain embodiments, the protein or peptide of the invention is considered an antibody because it binds to a target (e.g., a secreted protein or peptide or an extracellular protein or peptide). In one embodiment, the antibody comprises a heavy chain constant region, such as an IgG1, IgG2, IgG3, IgG4, IgA, IgE, IgM or IgD constant region. Preferably, the heavy chain constant region is an IgG1 heavy chain constant region or an IgG4 heavy chain constant region. Furthermore, the antibody can comprise a light chain constant region, either a kappa light chain constant region or a lambda light chain constant region. Preferably, the antibody comprises a kappa light chain constant region. Alternatively, the antibody portion can be, for example, a Fab fragment or a single chain Fv fragment.

In some embodiments, the peptide compositions of the invention decrease the level or activity (e.g., enzymatic activity, substrate binding activity, receptor binding activity, etc.) of the target secreted or extracelluar protein or peptide. The binding peptides of the invention include a variety of forms of antibodies including, for example, polyclonal antibodies, monoclonal antibodies, intracellular antibodies (“intrabodies”), Fv, Fab and F(ab)2, single chain antibodies (scFv), heavy chain antibodies (such as camelid antibodies), synthetic antibodies, chimeric antibodies, and a humanized antibodies.

In some embodiments, the invention provides a library of engineered heavy chain antibodies, or nanobodies. As with other antibodies of non-human origin, an amino acid sequence of a nanobody can be altered recombinantly to obtain a sequence that more closely resembles a human sequence, i.e., the nanobody can be “humanized” to thereby further reduce the potential immunogenicity of the antibody.

In some embodiments, the invention relates to the binding portion of an antibody or nanobody that comprises one or more fragments of an antibody or nanobody that retain the ability to specifically bind to binding partner molecule. It has been shown that the binding function of an antibody can be performed by fragments of a full-length antibody. Examples of binding fragments encompassed within the term “binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a FIT fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “binding portion” of an antibody. These antibody fragments are obtained using conventional techniques known to those with skill in the art, and the fragments are screened for utility in the same manner as are intact antibodies. Binding portions can be produced by recombinant DNA techniques, or by enzymatic or chemical cleavage of intact immunoglobulins.

### Nanobody Compositions

In some embodiments, the invention provides single chain antibodies, or nanobody polypeptides, that are directed against or can specifically bind to at least one secreted or extracellular protein or peptide, as well as compounds and constructs, (e.g., fusion proteins and polypeptides) that comprise at least one such amino acid sequence, and nucleic acid molecules encoding the nanobodies of the invention.

It should be noted that, in general, the term nanobody as used herein is not limited to a specific biological material or a specific method of preparation. For example, methods for preparing the nanobodies of the present invention include, but are not limited to, (1) isolation of a V_{HH} domain of a natural heavy chain antibody, (2) expression of a nucleotide sequence encoding a natural V_{HH} domain, (3) humanization of natural V_{HH} domains or expression of nucleic acids encoding the humanized V_{HH} domains, and (4) carnelization of natural V_{H} domains from any animal species, particularly mammals (eg, humans), or expression of a nucleic acid encoding a camelized V_{H} domain, (5) and synthesis of nanobodies or nucleic acids encoding nanobodies using amino acid or nucleic acid synthesis techniques. Suitable methods and techniques for carrying out the above will be apparent to those skilled in the art based on the disclosure herein, including, for example, the methods and techniques detailed below.

In some embodiments, the nanobodies of the present invention comprise an amino acid sequence that matches the amino acid sequence of a natural V_{HH} domain, but is “humanized” by substitution of one or more amino acid residues of the amino acid sequence of said native V_{HH} sequence with one or more amino acid residues occurring at corresponding positions of a V_{H} domain from a conventional human 4-chain antibody. The humanized nanobody of the present invention can be obtained by any suitable method known in the art.

In some embodiments, the nanobodies of the present invention are derived from a conventional 4-chain antibody by “camelization” (ie, substitution of one or more amino acid residues of a V_{H} domain with one or more amino acid residues occurring at corresponding positions in the V_{HH} domain of the heavy chain antibody). In some embodiments, the camelization occurs at the amino acid position present at the V_{H}-V_{L} junction and so-called Camelidae characteristic residues (see eg WO 94/04678). The camelized nanobody of the present invention can be obtained by any appropriate method known in the art.

In various embodiments, the invention provides nucleic acid molecules encoding the nanobodies, including humanized or camelized nanobodies, of the invention. is It can be carried out by expressing the nucleotide sequence thus obtained. In some embodiments, a nucleotide sequence encoding the humanized or camelized nanobody of interest of the present invention is designed, and the nucleic acid sequences thus obtained can be expressed in order to provide the nanobodies of interest of the present invention.

In one embodiment, the nanobodies of the invention binds to and, thereby partially or substantially alters at least one biological activity of the target (e.g., enzymatic activity, substrate binding activity, receptor binding activity, etc.).

In some embodiments, the invention includes compositions comprising an antibody or nanobody that specifically binds to a secreted or extracellular protein or peptide. In one embodiment, secreted or extracellular proteins or peptides include, but are not limited to GDPD1, METRN, PLA2G5, CD320, LGALS1, SCGB1A1, PRRG1, CEACAM7, SLC22A1, SCGB1C2, SPINK4, SIGLEC16, ZG16, LAMA3, TUSC5, LRRTM2, ENDOU, SV2C, TRPC3, GNRH2, OS9, FCGR2C, MMP24, FCAMR, EPHA7, MYORG, EDDM3A, FGF22, MST1, ACVRL1, GML, GPM6A, SLC6A8, RNFT2, FGFR2, S100A12, NODAL, CFHR1, FLJ36131, SPN, WFDC2, CCL24, MCP, ARSE, SCGB1D2, TRPC7, ODAPH, LIPM, MFAP4, HEPH, RNASE6, IL9, PRSS44, SLURP1, BAGE2, ABI3BP, CPA6, HP, TMEM206, INHA, C17orf77, ATP6AP2, GP2, CSH2, IFI30, FIBCD1, LDLRAD3, TSPAN9, AHSG, TSPAN2, TRPV1, LRIG2, IL18RAP, APOA1, IL28RA, IL1RAPL2, IER3, DPP10, BPIFA4P, SLC22A13, DEFB116, PTPNS1L2, PSORS1C2, TSPAN33, CD48, MOG, EFNA4, SERPINB2, SIGLEC15, MFAP3L, BPIFB1, NPTX2, HEXA, HAPLN3, FOLR2, TNFRSF19, SEMG2, PRRT1, S100A13, EXOC3-AS1, ENPP7, CD99L2, CNPY2, ACVR2B, CD164L2, LDLRAD2, TGFA, SCRG1, C14orf93, AMN, SBSPON, LMBRD1, AGRP, C5orf64, LRRC25, C22orf46, PAEP, IL25, EPHB2, PTPRA, POFUT2, TSHR, PNLIPRP1, VNN1, RTBDN, CLEC2D, NRN1, FRZB, NOG, TMPRSS11B, APOA2, NIPAL1, IFNA17, SPACA1, FLRT3, L1CAM, FZD7, GREM2, MMP15, VN1R2, MCFD2, FKBP9, C1orf54, SLC6A14, KDR, NPDC1, ILS, SCN4B, PRH1, KLK10, SLC22A10, GFOD2, ZPBP2, CGB3, IL27RA, CSN1S1, THBS3, DRAXIN, CD164, DEFB126, FAM19A4, UNQ6190/PRO20217, WFDC13, MMP13, TNFRSF11A, PTPRC, LINC00527, CLDN7, TPSD1, SEMA3E, GDF7, SCGB1D1, REG3G, CDH13, GABRB2, KLK15, FOLR1, AMIGO2, SLC6A2, PGA4, RNASE3, CHRNA4, SCARA5, KCNA6, CSN3, SCGB1C1, SLC22A4, CFP, UNQ6494/PRO21346, FCGRT, PSG9, TMEM41A, ABHD15, ROR1, ACKR2, POMC, MMP1, TNFRSF10D, IL3RA, NPC2, PSG4, TFF1, KCNMB3, CCL22, ULBP3, TIMD4, CD200R1, PCSK9, NXPH2, IL31RA, FP248, REG1B, LYPD1, CST9LP1, GYPA, ASTL, EMC10, FSHB, ANGPTL5, C11orf94, ZACN, CD55, CLDN1, APCDD1L, BSPH1, WFDC12, NGRN, TPBGL, GDF3, FKBP7, LYZL1, VASH2, TGFB2, CDCP1, GABRA5, CELA2B, SIRPB2, TNFSF8, UPK1B, PTCRA, CHRNB2, TMEM235, CSHL1, CCL27, APOF, SIGLEC8, FGF18, TMPRSS11D, TNFRSF13C, SLC22A25, UGT1A1, APOC2, SLCO2A1, CRTAC1, XCL1, FAM24A, PCSK2, FGF23, PTGDS, FAT2, ABCG2, LTA, CHRNA3, IGSF5, HSD11B1L, GDF11, KLRF1, THEM6, ENTPD5, CYR61, CES5A, RBP4, ANXA2P2, GM2A, SLC6A15, SCG5, CLDN14, C1R, SLC39A14, CST3, TNMD, SAA2, CHRNB4, NCAM1, NOV, PATE3, FAM3A, LAMP5, OGN, IFNA6, LRIT3, PANX3, ADM, FGF7, CSPG5, GFRA1, VWC2L, CCL15, MPEG1, CST5, TOR1B, LY6G5B, IL31, P4HB, CST7, OBP2B, PDGFD, DPEP2, GABRR3, LEG1, NTM, SPACA7, GNPTG, NPPC, ADAM30, OOSP1, IL17RD, TECTB, RNASE11, SECTM1, REG4, LAIR1, CA4, CRISPLD2, CCL16, CCL11, CTSW, UPK1A, CD9, LPAL2, SEMG1, IL17RA, CCL18, CNTN5, KRTDAP, BPIFC, LIPG, CCL8, MPZL1, CLPSL1, CST8, IL2RA, ECE1, F11, MSMP, KLK12, ACP4, CD59, PLGLA, EOGT, IL1RN, TNFRSF6B, PLA2G2A, ENTPD8, RNF128, FGFR3, FCGR3A, ABHD12, HBEGF, LRRC8B, MME, GPA33, IL5RA, TNFRSF10B, TFF2, NBL1, PROKR2, GUCY2F, CLPTM1L, AGER, ERBB2, DYNAP, IFNA5, DEFB132, TMEM158, AJAP1, IL6ST, SDF2L1, RAMP1, AMELX, NRN1L, EGFL3, SLC44A5, SSBP3-AS1, IL1RL1, CNGA3, IL17BR, CLEC-6, BTBD17, ZP4, NAALADL1, GLRA3, LY6G5C, CD300LG, NXPE3, IGF1, LGALS7, CCL4L1, SCARF2, PRTN3, LHFPL5, HTRA3, IFNGR2, PAP, MMP2, NRXN3, CALY, TEPP, B2M, TNFRSF19L, LRFN1, SCGB2A2, S100A8, ENSP00000381830, LY6G6F, SLC6A13, TMX2, IL22, C9orf47, MIA2, CFH, TIMP4, SRGN, PRND, PILRA, SFTA2, IL32, XG, ACVR2A, JTB, BMPR1A, CHRNA2, CLEC1B, SLC5A1, CHRNA9, VSTM2B, PSG6, CYTL1, FCGR1B, ADAM11, EPHA10, LOC348174, SERPINF2, CD70, TAS1R2, HLA-DRB3, FIGF, WISP3, LY6G6D, SLC6A9, MSLN, PPT1, EQTN, LHB, CD47, CD99, IFITM10, SPINK8, TEX46, MIA, TNFSF10, EVI2A, C6orf120, GABRA1, CHAD, IL8RB, KIR2DS2, AGPAT1, GZMK, TMEM169, PLGRKT, R3HDML, CRP, F7, CBLN4, KCNV2, IL10RB, IFNA8, OAS1, PRSS54, LCN9, C2orf40, HTR3C, SLC38A2, PCSK1N, PLXNA4, SPINK6, CTSD, FITM1, C2orf69, CHRNA1, SGSH, LY9, CD2, MCEMP1, RNF130, EPHA5, IL17F, FCN1, CSF3, CLPS, FLJ37218, SERPINB5, PRSS3, CXCL9, C9orf135, DIPK2B, C1QTNF2, ARSI, MASP1, GDF5, HTRA1, EMCN, LINC00305, NGFR, PRSS23, SERPINA7, PRRT3, HPR, SV2A, NAAA, SHISA8, PRSS2, ASIC1, DKK3, SLC29A4, FBLN5, MMP3, CXCL11, IL12RB2, ERVFRD-1, LY96, WNT16, CALR3, PROK2, SLC38A4, AXL, EDAR, GRIA1, LECT2, C6, FKBP11, GITR, FNDC4, CYP2D7, SERPIND1, RLN1, SERPINA9, LYNX1, C4A, CT83, LRG1, ABHD18, SLC39A12, GABRE, RNASE8, LSAMP, GABRA2, PDGFC, SLC6A1, DGAT2L7P, SLC22A8, CLDN17, PNLIP, IGSF21, FCRL1, ERP27, NAPSA, EFNA1, SLAMF6, PODN, OVCH1, Ly6L, SIGLEC9, FGF4, PF4V1, UPK3BL1, LMBRD2, LILRA5, IL8, PDIA2, PRNP, TNFRSF8, SLC2A2, HAMP, LY6D, C1orf32, TMEM108, CLDN20, LRRC8A, SLC6A7, TSPAN7, KIR2DS3, COL8A1, BTNL8, ICAM1, SERPINA13P, NRG4, SGCD, GLRA2, HEPACAM2, CST6, BTNL3, CSTL1, ANGPT4, DDR1, IL36B, S100A7, WFDC9, TMEM119, DPEP1, CLDN12, TSHB, F3, FGF1, NRG2, TM9SF1, FAM19A3, CDH2, TLR5, FAM3D, LYPD6B, ACP7, NTF3, CHGA, PIP, KIR2DS5, FAM24B, CFHR5, SLC34A3, CD6, ADAM32, RNF148, DCN, FGF21, RSPO4, SLC22A31, MMP27, FGF17, TGFB3, SLC15A1, GKN2, BST2, C5, CD8B2, SLC6A5, IGFBP5, FCRL6, LRRC3C, SIAE, IL1F5, C11orf45, SFTPA1, IZUMO4, IL22RA1, ACVR1, CD28, BTLA, CD302, GPHA2, LCN12, OSTN, FAM189A2, EPHA1, BTNL2, HDHD5, UNC5A, NOX1, BAGE3, CREG2, VEGFB, CBG, PRR27, CD244, HAPLN2, KIR3DS1, LPL, GYPC, P2RX5, GLT1D1, ARSJ, DNASE2B, PVR, CLPSL2, CD37, SMOC2, EDA2R, GABRQ, WISP2, FAM187B, SLC41A2, SPOCK1, KCNK1, DLK1, TPSAB1, IL1F6, MFAP2, STIM2, BCAN, SLC52A3, C19orf18, ATP1B2, TGFBR1, NPTN, LRP10, AND5, UMOD, TNFRSF7, RNF174, HLA-DRB4, PROCR, FGF19, OSM, RAET1L, CD83, ASIC4, ERVMER34-1, SELL, PRCP, SPINT2, CEACAM4, PVRL4, IZUMO, BVES, MFGE8, MICA, GASK1A, TMEM156, APOC3, MIF, ERVV-2, DLK2, SHBG, EPHB1, COLQ, CD163, NETO1, CLEC9A, PLXDC2, ERVK13-1, CLDN2, DEFA1, C1QR1, LHFPL1, ADAMTS15, PDCD6IP, BMPR2, GABRG1, NRTN, BTN3A3, CTSS, NCSTN, CDH26, CUTA, TM2D1, TRPC4, SLC6A20, ALB, SHISAL1, TIGIT, CLEC10A, CD163L1, ENPEP, APOD, KCT2, PTPRN2, CPA2, RNASE13, KLK13, KLK11, UNC5D, RSPO1, CD19, MMP28, FCER2, ILDR1, ENPP4, CD300E, UGT2A3, APCS, MUC5AC, CEACAM20, CLDN11, LCN2, ENPP5, SDC1, C9, CXCL1, SLCO1B1, ADAM2, SLC22A2, CD40LG, NHLRC3, INSL6, LDLR, LAMC1, CACNG4, IL28B, GAST, PROZ, UGT3A1, NCR3, ASTN2, VIT, OTOS, GGT7, AGT, PRSS3P2, SPINK14, CRLF1, CPN2, ICAM2, FAM19A2, SDCBP, LIFR, KLRG1, CD80, CD53, CA12, BMPR1B, SIGLEC7, RESP18, SEMA7A, PHEX, IGSF8, ASAM, MESD, TNFSF18, CSAG1, IGSF6, PDPN, SERPINI1, TRPC5, SMIM23, LCN6, CCL2, C10orf59, LYSMD3, CBLN3, RLN2, PCDHB1, BGN, TENM1, KISS1, GRIK3, FKBP14, IGSF4C, ITGA5, CTSK, CRELD1, CDH22, ICAM3, KLRC3, TNFRSF5, SLC5A8, FAM200A, TRABD2A, SHISA7, SCN3B, CFC1, CST1, LRRC8C, SIGIRR, MANF, CES3, CD34, KLK2, C1QL3, MFRP, IL7, DMP1, IL1A, SCN1B, TM2D3, OC90, LGALS3BP, HPN, REN, LRRN4CL, OVGP1, CLDN10, VSTM2A, A1BG, PAPLN, TSPAN31, SLC10A5, IFNE, LILRA4, DEFB113, C12orf49, METTL9, CHRNE, BTN2A1, SFTPD, PLAC1, SPAG11B, PEBP4, SYCN, ABCG1, THY1, IGF2, TNFRSF11B, CXCL5, NFAM1, DEFB136, IGFL1, NIPAL4, IHH, EVI2B, TBSP, CPED1, GKN1, CXCL2, BGLAP, KIR3DP1, VPREB1, KLK5, FST, CLEC12B, LIF, PRR4, PDIA6, LYZL6, DKFZp686O24166, CES4A, ATP1B3, EPGN, TNFRSF1A, VWC2, NXPE4, GPC1, TTYH3, TSPAN12, SLC10A4, CST4, SERPINA5, MINPP1, SLC6A11, NINJ1, THBD, TNFRSF10A, SPINK1, SIGLEC12, TMEM59L, ADCY5, CSMD1, KCNMB2, LRCH3, NTRK3, TMC4, KLK4, TMX1, TFPI, ANGPTL4, TEX101, TMPRSS4, EGFL7, ITPRIPL1, DEFA5, MUCL3, FCMR, LIPN, CSF2, CD72, RHBDD1, CLEC4A, ULBP2, CHRNB3, MUC1, GP6, DHRS4L2, TRH, SLC22A9, LSR, IL1F7, ADAM5, WNT7B, PLAUR, SOSTDC1, PTGS2, RS1, LRFN3, ADAMTSL5, IL2RB, TREM2, GPIHBP1, OPN1MW, ENG, IGSF10, GABRA4, MMP9, ANO6, GPHB5, DPT, IL11RA, APOO, SPACA3, TMEM182, LINGO3, PTN, CLEC19A, ACRV1, CXCL6, GUCA2A, GALNS, TMEM123, CLEC6A, ADAM15, TM7SF3, TULP1, BRINP3, FDCSP, ORM2, LCN15, VMP1, AMIGO3, CLDN8, FCRL4, SMPDL3B, PRRG3, WNT9B, MICB, ITLN2, SHISA4, INHBC, IGLON5, ARSK, TTR, FAM171A1, PPT2, SMO, PINLYP, SLCO1B3, RNASE9, SLC2A12, ADAM23, CES2, NAGPA, TSPAN3, PTX3, PCDHB7, SLCO2B1, PDZD11, CCL19, RNASE10, CTSV, PDILT, DKK1, MMP7, CLEC7A, CNPY4, MFSD2A, PRV1, SLC2A13, KLRK1, SLC1A1, FAM19A5, SMR3A, NT5E, TRABD2B, GLDN, ST14, IFNG, PIGR, PTHLH, CEACAM18, CD3D, FXYD5, BDNF, ENTPD2, RGMB, C1QL1, CTSG, PVRL1, LRFN2, MPZL3, CLEC17A, CADM2, PGF, KIRREL2, MOXD2P, SCGB3A1, LYPD8, EPCAM, KLRF2, SPP1, HSPA13, CES1P1, RAMP2, MLN, GABRG2, CST11, TNFSF13B, IGFBP6, NPPB, CNDP1, CD274, TMIGD1, PRADC1, SLAMF8, LYSMD4, SGCZ, LYZ, HS3ST1, LY6K, LY6H, LRRC32, GNLY, C6orf25, SCGN, FCN2, LOC644613, SSPN, AMY2B, FGFBP3, IL2RG, PCYOX1, GPC2, TNFRSF10C, CLDN18, SIGLEC10, NENF, CNTFR, PATE1, ICOSLG, IL29, CHGB, LY86, LRRC3B, SLC3A1, CA11, CECR9, P2RX2, CLDN16, SLC39A4, ROS1, TUFT1, STC2, SERPINE1, SPON2, GSG1L, EVA1C, TGFBR3L, CLDN19, CLDN9, BPIFA1, NTNG2, PSG2, HMSD, ASIC3, EBI3, SPINK13, ASIP, PRAP1, PGC, C1QTNF3, BMP2, LRRC26, FSTL3, IL15RA, GP1BB, PDCD1, COL26A1, SPINK9, TNF, C10orf25, IFNA14, ART1, ITGA6, CGA, PRSS8, BTC, GINM1, WNT4, CHRNG, DSC3, KCNMB4, MAG, SIRPA, SPATA20, LRTM1, TDGF1, C1RL, PLA2G2E, SLC4A2, INSL5, SDC4, DCD, CD226, TMIGD2, LRRC8D, RNASE12, ERP29, PGA3, BACE1, FBN2, COCH, SPOCK2, ENSP00000320207, RCN3, PCDHB5, TMEM130, WNT3A, RET, SLC22A6, OLFM2, CDH8, C1QBP, TMPRSS11F, PRSS55, C3, BY55, LGALS3, DDR2, NRROS, IDE, SOST, UGT8, SEMA3C, MARCO, SV2B, STC1, ENTPD3, LAIR2, GRN, GGT3P, GDF15, TNFRSF4, HHLA2, CTSH, AMHR2, PDCD1LG2, VNN3, IGLL5, CEACAM6, TREML1, SLC2A10, SCGB2B2, TM2D2, ASIC2, CSN2, LGMN, IGFBP7, MGP, DUOXA2, CGB7, TNFRSF6, FCN3, KLK3, EMB, TAS1R3, TMEM102, LCN1P1, FGF8, PMCH, IL4R, IL13, ASGR1, C1QTNF12, PRLR, OAF, PVRL2, APOE, TNFSF13, TNFSF9, CDH24, CLEC2A, SLC6A19, CHIA, LACRT, FAM3C, ICOS, ACKR1, SLC24A3, NTRK1, CDSN, TYROBP, CEACAM21, GUSB, FMR1NB, LUM, ESM1, RNASET2, KLK8, VSIG1, SLC1A5, VPREB3, MMP26, WFDC3, DAND5, SUSD3, SUMF1, TINAGL1, ODAM, TNFAIP6, ANGPTL8, DKK2, ATP4B, MANSC4, SPARC, IL33, SERPINC1, AZGP1, DNAJC10, EPYC, FGF9, IFNK, RDH5, SLC22A14, CFHR2, PNLIPRP3, TSLP, OXT, VMO1, IL1B, ERVFC1, ATP1B1, HCRTR2, IZUMO1R, MSMB, CST9L, BSG, UNQ5830/PRO19650/PRO19816, TNFSF15, AMBP, SIGLEC6, IFNL2, SSC4D, PSG5, IL1RAP, EMC7, FLT3LG, LYPD5, SERPINI2, TSPAN13, FLRT1, SIGLEC5, PRELP, EMR1, HYAL1, PLGLB1, LRRC4C, FURIN, P2RX1, PLA2G12B, NRXN1, WFDC8, SCARF1, SHISA5, SDC2, SDC3, POGLUT1, PLA2G2F, SLPI, PSCA, CNTN2, GGT1, UCMA, ATRAID, C17orf67, CNGA1, RAIVIP3, ITGB5, BMP5, RNF149, WISP1, MPO, CHRND, ADCK1, DNASE1L2, ERVK-18, PCDHGB6, HYAL2, CGB1, SEMA4D, TFPI2, C1QTNF6, BMP4, MMP11, SDF4, ANTXRL, CCK, OMD, GDPD3, HSD17B7, MDGA2, KLRC1, TMEM161A, P GLYRP1, C1QB, XK, P S APL1, C SF 2RA, OP CML, KLRC2, DCSTAMP, LHFPL6, SIGLEC11, IL18BP, UNQ6126/PRO20091, DPP6, ELFN1, OLFM1, KREMEN2, IL21, DNASE1L3, CLCA3P, ADM2, CD68, AGR3, NEGR1, TRPC1, AFP, SEMA3A, PRSS42, FAM174A, FCRL2, GPC4, UNC5C, TPBG, TNFRSF14, PLVAP, SLC34A2, RGMA, ECSCR, EFNB1, LCTL, CPAMD8, DKKL1, HPX, EREG, VEGFC, CLEC2B, IZUMO2, IGFBP3, SCGB2A1, TGFBR2, BCAM, MMP17, SGCB, JCHAIN, INHBE, COLEC11, PI3, FCRLM1, TCTN3, EMC1, NETO2, MUC21, CD3 G, RAET1E, CBLN2, PCSK1, JAG1, CLDN3, IL19, SPX, NTN3, CARTPT, ADAM7, UNQ9165/PRO28630, ASGR2, CDCP2, F1146089, ERVW-1, FCRL3, SOWAHA, WIF1, LRRC37B, GRM3, MUC13, SDF2, PLA2G12A, SCGB3A2, CD36, ERMAP, KLRD1, KLKB1, SFN, GABRA6, CCL20, PLBD2, PSG8, BMP10, VCAM1, PDGFB, SUSD6, VEGFA, EFNA5, NLGN4X, SYNDIG1L, PCDH10, FAM180A, IFNA13, PPBP, TREML4, CTSF, TMEM190, LALBA, HLA-DRB1, SFRP2, CGREF1, CTRB2, GPI, LRRN1, DNASE1, PCDHB3, PKDCC, PLA2G15, IL22RA2, WNT5B, GPLD1, HAPLN1, MPZL2, CCL21, C1QTNF7, CPA5, CLTRN, PLA2G10, ALKAL1,INS, IFNW1, NUCB1, OBP2A, PRNT, FUCA1, ENH3, TMEM8B, CFD, AGPAT2, PCDHB9, CDH2O, ADIPOQ, MFAP5, RSPO2, IL16, F11R, SLC39A5, CHI3L1, PLA2G2D, C4BPA, GDF2, CTSA, SNCA, PRL, TMEM9, BMP6, FLT3, UGT2A1, RSPO3, KIR3DX1, FETUB, ABHD17B, SORT1, LGALS9, CADM1, FAM198B, FSTL1, FAM20C, CCL25, OIT3, TMEM9B, LY6G6C, F13B, ABCG8, SPACA6P, IDS, PGLYRP3, RXFP1, CD300LD, KIR2DL4, TXNDC15, OPN4, FAM187A, VTN, IL20, NLGN4Y, SRPX2, TMPRSS11A, KLK9, FSHR, TCTN2, PCYOX1L, SLCO4A1, LOXL4, GZMB, OOSP2, LYPD3, PAMR1, MUC7, ART4, CLU, TXNDC12, LLCFC1, CD69, CTHRC1, CTLA4, HAVCR2, WFIKKN1, TLR6, GDF8, GPX7, S100A9, EFNA2, FCRL5, KLRC4, SFRP4, BMP8A, SGCE, TMEM149, SLC1A4, LRRC4B, CD200, GNRH1, ADAMTS16, COL14A1, LRRN5, LCN10, FLT1, PLA2G3, CSF3R, HTR3B, GP9, LRFN5, FIBIN, MMP8, CXCL17, PRSS50, CDH5, CXCL10, ZP2, GLB1, DEFB135, MEPE, TSPAN1, APP, ELFN2, KIR2DS4, C7, TNFRSF21, WNT7A, FKBP10, TMEM204, GRIA3, AVP, IL21R, CD300LB, FCAR, TMED1, CALR, PDGFA, ADAMDEC1, CHRDL1, FKBP2, C OLEC10, PO STN, TNF SF14, C11orf24, ERVK-7, SPINK7, ADGRE3, TNFRSF17, FGF20, SAA4, C1QTNF5, TWSG1, SLC22A20P, MFAP3, BMP3, BTN2A2, HPSE, PRDX4, PTPRJ, PKD2, CEACAM1, CHRNA6, ENPP3, ARTS, FZD10, TMEM25, EDDM3B, LIPK, ITGB8, C2orf66, SNORC, TGFBI, ITLN1, SERPINA1, CRTAP, TEX264, UNCSB, SFTA3, RETNLB, AQP10, CCL3, NRP1, SERPINF1, SPAG11A, DEFB112, TMPRSS13, PLA2G7, LINGO2, IGDCC3, TMEM81, KSP37, IFNGR1, CFB, ITGB1, KITLG, SCARA3, TIMP2, ROR2, GATD1, LRIT2, ZP3, CD86, LY75, PRSS58, PON1, PSPN, FOXRED2, RCN2, FNDCS, SLAMF9, HAVCR1, APOM, GABRD, DUOXA1, APMAP, TSPAN8, HRG, TAC4, NCR1, KIR2DL5B, FGFRL1, RETN, ERVH48-1, NRG1, GBA, CCL7, TSKU, VSTM5, NDP, ROBO2, CLEC4C, CDNF, AMY1A, CD44, SEMA4F, GSG1L2, NTF5, FNDC7, C5orf38, CXCL3, IL6, EFEMP2, BTNL9, NGFB, GPR108, PLAT, CD300LF, DNAJB9, NXPH1, C2, PCDH12, DEFB108B, CNPY3, ARSG, WNT8A, IL4, IL1R2, VSTM4, PNLIPRP2, AGRN, IL9R, PLA2G1B, BTNL10, CHIT1, CCR9, ANO4, PIANP, EDEM2, SFRP1, GDF10, CCL1, SHISA9, IL17RC, BTN1A1, CRELD2, LYG1, CD74, LRTM2, PGLYRP4, CCL23, TMEM91, TTYH2, PGLYRP2, CCDC126, C1QTNF1, SEMA3B, LYPD2, BPIFA3, PTPRG, IMPG1, CRISP1, KAL1, CTF1, SFTPA2, PPIA, CD248, NAGA, DNAJC3, C1QL2, VSIG8, C1orf233, KCNMB1, NTRK2, CELA1, ADAM19, IGFBP4, AMTN, SEMA3D, IGFL4, GVQW1, CX3CL1, C1QTNF9B, VASN, TMEM59, LILRA3, CA6, EPPIN, TMEM132A, KLK6, RTN4R, AKR1B10, CD207, CPE, INHBB, SLC22A5, FGB, LGI1, CHID1, PTPRR, TM4SF6, CD300A, NOPE, EFNB2, IGF2R, FAM3B, SMOC1, TMX4, NINJ2, LHCGR, IGIP, ENH1, IL15, PTCH2, CR1L, TPP1, PROK1, CCBE1, RNPEP, SPP2, IL13RA2, HSD17B13, SFTPB, CASQ1, CD96, NPNT, LILRB1, MXRA8, LEP, CDHR2, DNASE2, CA14, F2, VSIG4, GGH, OTOR, WFDC10A, FGF5, IGSF11, FSTL5, NOX5, GLRA1, BMP15, FGG, LRRC52, FGFR1, LRRTM1, TMCO3, LRP12, MPL, MEP1A, LRRC4, EPHA8, METTL7A, CCL17, IGLL1, FAM234A, CDH10, GABRA3, LINGO4, CCDC134, DHRS7, GDF1, RSPRY1, KIR3DL3, FJX1, GP5, PRSS57, WNT5A, PCDHB6, MMP10, TXN, C19orf10, TNFRSF1B, JAM3, CHRNA5, SLC34A1, EPHA2, PCOLCE, HAPLN4, DHRS7C, IL34, SGCA, IL6R, PTTG1IP, TREML2, CCDC70, CDH19, CLCC1, AMBN, BTN2A3P, SLC8B1, TMEM154, NUCB2, ADCYAP1, ERVK-24, GHR, CFHR4, ALPL, C1QC, CPO, C11orf44, OVCH2, GPX5, TOR2A, SHISA6, CCL28, CD84, NXPH3, LRRC55, CRLF2, RCN1, ASIC5, SLC22A11, XCL2, LYPD6, DAG1, LILRB2, TNFSF6, MMP25, CDH1, BAMBI, KLK14, SCN2B, FOLR3, TSPAN15, C21orf62, LAG3, HGFAC, CEACAM19, ADAMTS18, CHI3L2, TINAG, AMY2A, CLEC4E, SPESP1, LRCOL1, GFRAL, EPO, P2RX3, GABRB3, SIRPB1, CPVL, CD151, IL17A, HABP2, KAZALD1, EGFLAM, NCR2, PRG3, CD14, SLC20A1, IGFL2, ERLEC1, C4BPB, LILRB4, ADGRE4P, TMEM95, THNSL2, IFNAR2, PM20D1, HPSE2, RNF43, IL13RA1, DLL1, GFRA3, LTBR, IL17C, TFRC, RARRES2, ZPBP, LCAT, VSIG2, CGB2, IL3, ACVR1B, TNA, CLEC1A, ARSD, PNLDC1, CXCL13, AMICA1, THBS2, MZB1, ANGPTL3, IZUMO3, ATP1A1, ADAM9, PECAM1, IL2ORB, IL1F9, UGT1A9, CPM, CD63, CMA1, CNNM4, PRSS21, WNT6, CXCL16, CYB5D2, SLC5A4, RNF167, SERPINAll, MAGT1, LINGO1, TLR3, NPR3, SLIT3, DNASE1L1, GBP1, ARSF, IL2, OTOL1, MR1, MMRN1, MUC15, OPTC, PLET1, CLEC5A, CBA, CD3E, FCGR2A, SPACA5, TNFSF11, BMP1, LCN1, CDH9, AMIGO1, SLC26A2, P2RX6, SHISA2, ULBP1, OLR1, GIF, WNT8B, FAM19A1, C8B, CHODL, SPACA4, CER1, SGCG, PATE2, CRISPLD1, CDH17, CDH15, NAXE, TLR2, SLAMF7, P2RX7, TMEM8, FCRLB, CCL13, TULP3, GDF9, SULF2, PTX4, LGI3, ANO10, GALP, FAM168B, ALPI, FGL2, TIMP3, ANO2, SERPINA4, WNT2, EFNA3, SERPINA2, CEACAM8, INSL3, PCDHGA4, CD81, GLRA4, EDIL3, ALPPL2, PATE4, CD4, LGR5, TGFB1, IL7R, APOC1, FZD4, FCER1A, CD5L, P2RX4, KERA, LYG2, HYAL4, PTH, C3orf58, ERP44, ISLR, CASQ2, CXCL12, ERVV-1, NTS, TARM1, P4HA1, ADAM28, KLRB1, NCAM2, LRRC15, TMEM155, SEMA6C, SERPINA12, ANGPTL1, SEMA6A, SERPINA3, HEPACAM, C1orf159, PF4, TCN2, MBL2, MEGF10, PRRG4, IL23R, HTR3A, LIPA, IGFL3, LOX, CD200R1L, IGSF4B, SELE, LAS2, C17orf99, IGFALS, COLGALT2, ZG16B, CBLN1, DGCR2, PCDHGC3, TMEM178A, VSTM2L, CNTN4, ACP5, TNXB, CEACAM16, CD38, GZMA, TPSB2, IL17B, KEL, IFNA2, CD5, LILRB3, SLAMF1, TEK, PCDHB16, SPINK5, TNFRSF12A, VLDLR, PDGFRB, HLA-DRB5, CRTAM, GFOD1, LRRC38, MATN1, MMP14, MUSK, IL1F10, LRRC21, GDPD5, REG1A, FGL1, EPOR, GNS, C1QA, ANTXR2, SLCO3A1, ENTPD1, ADAM29, PLBD1, HLA-DOB, LAMB3, IL1RAPL1, GDPD2, ITGA8, SRPX, CASP4, PI16, EPDR1, PROS1, ABHD17A, SCUBE2, CLEC4F, CREG1, ATP6AP1, CLEC4G, PRS S47, EFEMP1, CE Sl, CFC1B, TSPAN18, CD33, ITGA2, GABRG3, GH1, KIRREL3, C15orf61, GABRP, HEXB, HRC, GLIPR1, VWA2, PCDH2O, NOTUM, LRRC24, ITFG1, LRRTM4, COL10A1, SLC39A8, PVRL3, PRSS41, KCNE4, WFIKKN2, SMPDL3A, DPEP3, CDH23, IGFBP2, PCDHA9, FAM172A, HSPA5, GAS1, TMPRSS2, IFNB1, PTPRD, NLGN1, UGT1A3, CILP2, CD58, MFI2, TNFRSF9, P SAP, ENDOD1, APLP1, IL17RE, C1QTNF4, CPB2, TLR1, KIRREL, ECM1, SPARCL1, COMT, CALU, CST9, CLUL1, NLGN3, MUCL1, LRFN4, CCL26, TRIL, CD209, TMPRSS3, LGR8, CRISP3, METRNL, WNT9A, FGF6, UGT1A6, THPO, MMP21, ISLR2, COLEC12, DLL4, CDH18, TMEM120A, TMEM219, C9orf72, OLFML3, ALCAM, EPHB6, ITIH3, SERPINA6, DHRS13, FN1, CHRDL2, CNTN6, KLK1, LGR6, NDNF, ERO1B, CRISP2, DKK4, C10orf54, DEFB129, LRRTM3, ELSPBP1, ASPN, ADGRG7, GFRA2, FAM132B, CLEC4M, LGI2, PLA2G2C, CCDC47, AOC2, ZPLD1, MDK, QPCT, AGA, RTN4RL2, PKD2L1, BTD, LILRA1, PRG2, CNTN1, VNN2, UGT2B28, PRSS37, PILRB, CD300C, NPIPB7, FAM171B, DHRS9, PZP, IGFBP1, ESAM, ADGRE2, CDH16, NOTCH2NL, IFNAR1, IL1R1, MATN2, TMEFF1, TM9SF3, APCDD1, NEU1, ANXA1, FGFBP1, GDNF, CNTN3, ITGAM, CTSZ, AIMP1, METTL24, COMP, CST2, PRSS22, SCG3, CFI, CEACAM5, IGFBPL1, INFL4, GPC6, LTF, PLTP, FBN3, IL12RB1, CDH6, C6orf89, FMOD, CPXM2, ISM2, PRSS48, LILRB6, MSR1, SEMA4C, SIRPG, CD276, GDF6, PROC, TREH, SPON1, C17orf80, AGR2, SLCO4C1, NXPE1, PODNL1, PCDH9, PDIA3, RTN4RL1, F9, SIDT2, FLRT2, GSN, WFDC1, PLAC9, SCG2, F12, NTNG1, GHDC, MMP12, SPINT4, MAN2B1, TIMP1, NXPH4, LIPC, SOD3, EGFL6, IGSF9, NID1, ENPP6, APOA4, GC, COL9A3, HTR3E, CLDN23, ARTN, CSF2RB, HYAL3, TULP2, TACSTD2, GPC5, CPA1, SLITRK4, CLDN4, SLITRK3, ADAMTS17, ANGPT2, DLL3, GZMH, PRSS35, IL23A, LAD1, ADAM21, ANGPTL6, LIPF, C7orf69, OLFM3, IL2ORA, UGT2B10, GAA, FZD9, DPP7, CLEC14A, CHL1, CIS, LGI4, SCARB1, BCHE, CLEC3A, ITGB7, PCOLCE2, CALHM1, BOC, SEMA4B, CP, GABRB1, NPIPB15, CLSTN1, HNRNPA2B1, SLITRK5, PCDHB15, CNTNAP4, APOH, INSR, ABHD2, CXCL14, OLFML1, LRPAP1, DSC1, CLN5, IGSF1, TLR7, IL10RA, GP1BA, BRICD5, GCG, BPIFA2, FGF16, CEACAM3, BMP8B, APLP2, TSPAN17, LIPI, WNT3, SERPINH1, SIGLEC14, ERO1A, GABRR2, P3H3, LRP5, CLCF1, IL17D, PI15, GZMM, APOA5, LEPR, NOX4, SERPING1, IMPG2, LRP11, F5, CNTNAP2, LYZL4, TMEM178B, SLIT2, SCNN1B, CETP, VTCN1, PTPRF, OTOA, IL1RL2, SERPINE2, TNR, TGOLN2, CDH3, TOR3A, TP53I13, NAMPT, UGT1A8, ART3, BST1, OXNAD1, CPB1, EDA, MADCAM1, CHRNB1, OSMR, NCAN, PLA1A, GGT5, ITGB6, ITGAX, MEGF9, DEAF1, CD7, ITGB2, EFNB3, CRHBP, TLR9, BPIFB3, CLEC11A, TREM1, LEFTY2, TSPAN5, AOC3, LYPD4, CD180, UGT3A2, ELANE, LRRN4, DDRGK1, CDON, ATP1A3, PCDHGB3, CELA3A, HTR3D, DEFB128, PTPRO, LRIG3, TF, WNT11, HTRA4, NTNS, ERBB3, ATP2C2, IL10, LY6E, IL4I1, TLR4, ICAM4, TMPRSS12, PCDHB11, HLA-F, PRSS33, MRC1, LAYN, FBLN1, TCTN1, PROKR1, EPHA4, PRSS1, ADAM20, ANTXR1, SELPLG, NFASC, APOC4, ASPRV1, TMC3, CCLS, NRP2, CILP, IGF1R, SEC11B, AREG, ECM2, TFR2, CD79B, ACVR1C, CTRL, MEP1B, LYVE1, TMPRSS11E, F13A1, TYRO3, ADAM22, RNASE1, NID2, C6orf15, PDGFRA, NELL1, GLRB, FREM1, MMP19, ENAM, CPQ, MET, ITGBL1, TIE1, PODXL, PRTG, FAM20A, CLEC12A, LRRC19, CLCA4, HSD17B6, CDT6, SLC4A7, ITGA2B, EGFR, DNAJB11, DCST1, GREM1, JAG2, SCPEP1, HIDE1, ADAM18, KLK7, SPATA6, HHIP, KIR3DL2, CTSE, BACE2, LUZP2, CPN1, C14orf37, CLCA1, IL26, SLC5A5, LRRN3, SELP, P4HA2, MDGA1, ASTN1, CTSC, AZU1, SCUBE1, BPI, MYOC, KIAA0319L, CELA3B, NPTX1, CNTNAP5, and SNED1. In one embodiment, the secreted or extracellular protein or peptide is a target provided in Table 2. In one embodiment, the secreted or extracellular protein or peptide is CCL16, CD302, CD3D, CD3E, CEACAM4, ILlORA, IL13, IL17A, IL1RN, IL21, PROCR, TIGIT, TNF, TNFRSF17, or TNFSF18.

In some embodiments, a nanobody that binds to a secreted or extracellular protein or peptide of the invention inhibits, blocks, or interferes with at least one activity of the secreted or extracellular protein or peptide (e.g., enzymatic activity, substrate binding activity, receptor binding activity, etc.), in vitro, in situ and/or in vivo.

In one aspect, this disclosure provides an isolated antibody, or binding portion thereof, comprising a heavy chain variable region comprising at least one, two or all three CDR sequences of a SEQ ID NO selected from the group consisting of SEQ ID NO:1-SEQ ID NO:42890 as defined by the CDR flanking regions as set forth in SEQ ID NO:42891 and SEQ ID NO:42892 flanking CDR1, SEQ ID NO:42893 and SEQ ID NO:42894 flanking CDR2 and SEQ ID NO:42895 and SEQ ID NO:42896 flanking CDR3 for each of SEQ ID NO:1-SEQ ID NO:42890. Accordingly, in some embodiments, the invention comprises a nanobody comprising at least one, two or all three CDR sequences of a SEQ ID NO selected from the group consisting of SEQ ID NO:1-SEQ ID NO:42890 as defined by the CDR flanking regions as set forth in SEQ ID NO:42891 and SEQ ID NO:42892 flanking CDR1, SEQ ID NO:42893 and SEQ ID NO:42894 flanking CDR2 and SEQ ID NO:42895 and SEQ ID NO:42896 flanking CDR3 for each of SEQ ID NO:1-SEQ ID NO:42890.

In one aspect, this disclosure provides a nucleic acid molecule comprising a nucleotide sequence encoding an isolated antibody, or binding portion thereof, comprising a heavy chain variable region comprising at least one, two or all three CDR sequences of a SEQ ID NO selected from the group consisting of SEQ ID NO:1-SEQ ID NO:42890 as defined by the CDR flanking regions as set forth in SEQ ID NO:42891 and SEQ ID NO:42892 flanking CDR1, SEQ ID NO:42893 and SEQ ID NO:42894 flanking CDR2 and SEQ ID NO:42895 and SEQ ID NO:42896 flanking CDR3 for each of SEQ ID NO:1-SEQ ID NO:42890. Accordingly, in some embodiments, the invention comprises a nucleic acid molecule comprising a nucleotide sequence encoding a nanobody comprising at least one, two or all three CDR sequences of a SEQ ID NO selected from the group consisting of SEQ ID NO:1-SEQ ID NO:42890 as defined by the CDR flanking regions as set forth in SEQ ID NO:42891 and SEQ ID NO:42892 flanking CDR1, SEQ ID NO:42893 and SEQ ID NO:42894 flanking CDR2 and SEQ ID NO:42895 and SEQ ID NO:42896 flanking CDR3 for each of SEQ ID NO:1-SEQ ID NO:42890.

The antibody CDR sequences of the nanobodies set forth in SEQ ID NO:1-SEQ ID NO:42890, as defined by the CDR flanking regions as set forth in SEQ ID NO:42891 and SEQ ID NO:42892 flanking CDR1, SEQ ID NO:42893 and SEQ ID NO:42894 flanking CDR2 and SEQ ID NO:42895 and SEQ ID NO:42896 flanking CDR3, establish a novel family of secreted or extracellular protein binding proteins, isolated in accordance with this invention, and comprising polypeptides that include the CDR sequences listed. To generate and to select CDR's of the invention having binding and/or detection and/or inhibitory activity, standard methods known in the art for generating binding proteins of the present invention and assessing the binding and/or detection and/or inhibitory characteristics of those binding protein may be used, including but not limited to those specifically described herein.

In some embodiments, the invention comprises a nanobody comprising an amino acid sequence selected from the group consisting of SEQ ID NO:1-SEQ ID NO:42890. In some embodiments, the invention comprises a nucleic acid molecule comprising a nucleotide sequence encoding a nanobody comprising an amino acid sequence selected from the group consisting of SEQ ID NO:1-SEQ ID NO:42890.

The following is a non-limiting example of the compositions of the invention. The following is provided as an example of the use of the CDR sequences as defined by the flanking regions as set forth in SEQ ID NO:42891 and SEQ ID NO:42892 flanking CDR1, SEQ ID NO:42893 and SEQ ID NO:42894 flanking CDR2 and SEQ ID NO:42895 and SEQ ID NO:42896 flanking CDR3 to identify the compositions of the invention, and is not limiting as the invention should be construed to include similar compositions for each of SEQ ID NO:1-SEQ ID NO:42890.

In one embodiment, the composition comprises an antibody, or fragment thereof, for binding to CCL16. In one embodiment, the composition comprises a nucleic acid molecule encoding an antibody, or fragment thereof, for binding to CCL16. In one embodiment, the antibody, or fragment thereof, for binding to CCL16 has a binding score of at least 0.9.

In one embodiment, the antibody, or fragment thereof, for binding to CCL16 comprises at least one, at least two, or each of a heavy chain CDR1 sequence comprising amino acid residues 26-32 of SEQ ID NO:3779, a heavy chain CDR2 sequence comprising amino acid residues 46-57 of SEQ ID NO:3779, and a heavy chain CDR3 sequence comprising amino acid residues 96-105 of SEQ ID NO:3779. In one embodiment, the antibody, or fragment thereof, for binding to CCL16 comprises an amino acid sequence as set forth in SEQ ID NO:3779.

In one embodiment, the invention provides a nucleic acid molecule comprising a nucleotide sequence encoding an antibody, or fragment thereof, for binding to CCL16 comprising at least one, at least two, or each of a heavy chain CDR1 sequence comprising amino acid residues 26-32 of SEQ ID NO:3823, a heavy chain CDR2 sequence comprising amino acid residues 46-57 of SEQ ID NO:3823, and a heavy chain CDR3 sequence comprising amino acid residues 96-109 of SEQ ID NO:3823. In one embodiment, the nucleic acid molecule comprises a nucleotide sequence encoding an amino acid sequence as set forth in SEQ ID NO:3823.

In some embodiments, the secreted or extracellular protein binding molecules (e.g., antibodies, etc.) of the present invention, exhibit a high capacity to detect and bind their target, as indicated in Table 2, in a complex mixture of salts, compounds and other polypeptides, e.g., as assessed by any one of several in vitro and in vivo assays known in the art. The skilled artisan will understand that the secreted or extracellular protein binding molecules (e.g., nanobodies, etc.) described herein as useful in the methods of diagnosis and treatment and prevention of disease, are also useful in procedures and methods of the invention that include, but are not limited to, an immunochromatography assay, an immunodot assay, a Luminex assay, an ELISA assay, an ELISPOT assay, a protein microarray assay, a Western blot assay, a mass spectrophotometry assay, a radioimmunoassay (MA), a radioimmunodiffusion assay, a liquid chromatography-tandem mass spectrometry assay, an ouchterlony immunodiffusion assay, reverse phase protein microarray, a rocket immunoelectrophoresis assay, an immunohistostaining assay, an immunoprecipitation assay, a complement fixation assay, FACS, a protein chip assay, separation and purification processes, and affinity chromatography (see also, 2007, Van Emon, Immunoassay and Other Bioanalytical Techniques, CRC Press; 2005, Wild, Immunoassay Handbook, Gulf Professional Publishing; 1996, Diamandis and Christopoulos, Immunoassay, Academic Press; 2005, Joos, Microarrays in Clinical Diagnosis, Humana Press; 2005, Hamdan and Righetti, Proteomics Today, John Wiley and Sons; 2007).

More preferably, the secreted or extracellular protein binding molecules (e.g., nanobodies, etc.) of the present invention, exhibit a high capacity to reduce or to inhibit an activity of their target as indicated in Table 2 (e.g., enzymatic activity, substrate binding activity, receptor binding activity, etc.) as assessed by any one of several in vitro and in vivo assays known in the art.

As used herein, a secreted or extracellular protein binding molecules (e.g., nanobody, etc.) that “specifically binds to a target protein” is intended to refer to a secreted or extracellular protein binding molecules (e.g., nanobody, etc.) that binds to a target protein of any animal. In some embodiments, that antibody binds to a human secreted or extracellular protein.

In some embodiments, the secreted or extracellular protein binding molecule (e.g., nanobody, etc.) is predicted to bind to its target protein with a binding score of at least 0.5, at least 0.6, at least 0.7, at least 0.8, at least 0.9, at least 0.91, at least 0.92, at least 0.93, at least 0.94, at least 0.95, at least 0.96, at least 0.97, at least 0.98, at least 0.99 or greater than 0.99.

In some embodiments, the secreted or extracellular protein binding molecule (e.g., nanobody, etc.) binds to its target protein with a KD of 1×10⁻⁶ M or less, more preferably 1×10⁻⁷ M or less, more preferably 1×10⁻⁸ M or less, more preferably 5×10⁻⁹ M or less, more preferably 1×10⁻⁹ M or less or even more preferably 3×10⁻¹⁰ M or less. The term “does not substantially bind” to a protein or cells, as used herein, means does not bind or does not bind with a high affinity to the protein or cells, i.e., binds to the protein or cells with a KD of greater than 1×10⁶ M or more, more preferably 1×10⁵ M or more, more preferably 1×10⁴ M or more, more preferably 1×10³ M or more, even more preferably 1×10² M or more. The term “KD”, as used herein, is intended to refer to the dissociation constant, which is obtained from the ratio of Kd to Ka (i.e., Kd/Ka) and is expressed as a molar concentration (M). KD values for a secreted or extracellular protein binding molecule (e.g., nanobody, etc.) can be determined using methods well established in the art. A preferred method for determining the KD of a binding molecule (e.g., antibody, etc.) is by using surface plasmon resonance, preferably using a biosensor system such as a Biacore® system.

As used herein, the term “high affinity” refers to a nanobody having a KD of 1×10⁻⁷ M or less, more preferably 5×10⁻⁸ M or less, even more preferably 1×10⁻⁸ M or less, even more preferably 5×10⁻⁹ M or less and even more preferably 1×10⁻⁹ M or less for a target binding partner molecule. However, “high affinity” binding can vary between antibody isotypes. For example, “high affinity” binding for an IgM isotype refers to an antibody having a KD of 10⁻⁶ M or less, more preferably 10⁻⁷ M or less, even more preferably 10⁻⁸ M or less.

### Multiparatopic Antibodies

In some embodiments, the present invention provides amino acid sequences and antibody compositions that are capable of binding to two or more different antigenic determinants, or epitopes. In this context, the amino acid sequences and polypeptides of the invention are also referred to as “multiparatopic” (such as e.g. “biparatopic” or “triparatopic”, etc.) amino acid sequences and polypeptides. The multiparatopic amino acid sequences and polypeptides of the invention can be directed against any antigenic determinants, or epitopes. For example, and generally, a biparatopic polypeptide of the invention may comprise at least one amino acid sequence or nanobody directed against a first antigenic determinant or epitope, and at least one amino acid sequence or nanobody directed against a second antigenic determinant or epitope different from the first antigenic determinant or epitope. In some embodiments, the amino acid sequences and/or nanobodies are linked, for example via a suitable linker.

A triparatopic polypeptide of the invention may comprise at least one further amino acid sequence or nanobody of the invention directed against a third antigenic determinant or epitope, different from both the first and second antigenic determinant, epitope, part or domain.

In some embodiments, multiparatopic polypeptides of the invention may contain at least two amino acid sequences or nanobodies of the invention directed against at least two different antigenic determinants or epitopes of the same secreted or extracellular protein or peptide. In some embodiments, multiparatopic polypeptides of the invention may contain at least two amino acid sequences or nanobodies of the invention directed against at least two different antigenic determinants or epitopes of at least two different secreted or extracellular proteins or peptides.

### Generation Of Nanobodies

The invention provides a method of generating nanobody compositions that bind to a target extracellular or secreted protein with high affinity. In one embodiment, the nanobodies of the invention can be generated by using a high-throughput screening technique (HAPPY) that combines two technologies in directed evolution: yeast surface display and phage display.

In one embodiment, the method comprises the steps of panning phage-displayed nanobodies against a library of displayed extracellular antigens, eluting the phage using a protease to cleave the nanobody-bound antigens off the dusplay surface, and quantifying the degree of enrichment of individual nanobodies using next-generation sequencing.

In one embodiment the nanobodies of the invention are displayed as a phage library, where that phage comprises human immunoglobulin genes and the library expresses human antibody binding domains as, for example, nanobodies, single chain antibodies (scFv), as Fab, or some other construct exhibiting paired or unpaired antibody variable regions (Vaughan et lo al. Nature Biotechnology 14:309-314 (1996): Sheets et al. PITAS (USA) 95:6157-6162 (1998)); Hoogenboom and Winter, J. Mol. Biol., 227:381 (1991); Marks et al. J. Mol. Biol., 222:581 (1991)). Such phage display methods for isolating human antibodies are established in the art. See for example: U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

Preparation of a display library of secreted proteins, extracellular proteins, peptide fragments thereof, or any combination thereof can be performed using any suitable technique. Display techniques such as yeast display (Boder, Raeeszadeh-Sarmazdeh et al. 2012; Bosma, Rink et al. 2019; Linciano, Pluda et al. 2019) and bacterial display (Samuelson, Gunneriusson et al. 2002; Lofblom 2011; Shivange and Daugherty 2015), in which a polypeptide is genetically fused or non-covalently tethered to an outer membrane protein of the yeast or bacterial cell, has enabled the screening of large combinatorial libraries of linear peptides (e.g., 10⁶ to 10⁹ members) from which peptide ligands for a target molecule of interest could be identified (Shivange and Daugherty 2015; Linciano, Pluda et al. 2019). In one embodiment the secreted proteins, extracellular proteins, peptide fragments thereof, or any combination thereof are displayed as a yeast library.

In some embodiments, other cell types can be used to display secreted or extrecellular proteins. In addition to yeast and bacterial cells, mammalian cells, insect cells, and plant cells could be used. In some embodiments the yeast cell is from the species Saccharomyces cerevisiae, Schizosaccharomyces pombe, or Pichia pastoris, or Kluyveromyces lactis. In some embodiments the bacterial cell is from the species Escherichia coli or Bacillus subtilis. In some embodiments the insect cell is derived from the species Spodoptera frupperda, Trichoplusia ni, or Drosophila melanogaster. In some embodiments the mammalian cell is derived from the species Homo sapiens, Mus musculis, or Cricetulus griseus. In one embodiment the secreted proteins, extracellular proteins, peptide fragments thereof, or any combination thereof are displayed as a yeast library.

The isolated nucleic acids of the present invention can be made using (a) recombinant methods, (b) synthetic techniques, (c) purification techniques, or combinations thereof, as well-known in the art. DNA encoding the nanobodies is readily isolated and sequenced using methods known in the art. Display techniques, such as phage display, wherein the coding sequence and the translation product are linked, allow for simplified selection of an antibody or nanobody that binds to a target and isolation of the nucleic acid. After phage selection, the antibody coding regions from the phage can be isolated and directly sequenced, used to generate whole nanobodies, including humanized nanobodies, or any other desired binding fragment, or expressed in any desired host, including mammalian cells, insect cells, plant cells, yeast, and bacteria.

### Machine Learning Methods of Identifying Nanobodies with High Binding

In some embodiments, machine learning algorithms or general linear models are used to produce a predictor of target binding, or a binding score. Exemplary maching learning algorithms that can be used to generate a binding score, include, but are not limited to, logistic regression models, random forest analysis, support vector machines, neural networks, naive Bayes classifier, and nearest neighbour algorithms. The model is a multi-class classifier based on a logistic regression model.

In one emboidment, the binding score provides categorical values indicating the probability of whether a nanobody 1) binds to its target antigen via ELISA, but not a negative control antigen BSA; 2) binds to both the target antigen and BSA by ELISA; or 3) binds to neither target antigen or BSA by ELISA. In one emboidment, the binding score provides, the probability of a given nanobody being able to bind to target antigen, but not the control antigen, in ELISA format.

Factors which may be used as inputs to a machine learning model include, but are not limited to, percentage of NGS read count of a nanobody compared to the total read count of all nanobodies in Round 1 input, percentage of NGS read count of a nanobody compared to the total read count of all nanobodies in Round 1 output, percentage of NGS read count of a nanobody compared to the total read count of all nanobodies in Round 2 input, percentage of NGS read count of a nanobody compared to the total read count of all nanobodies in Round 2 output, percentage of NGS read count of a nanobody compared to the total read count of all nanobodies in Round 3 input, percentage of NGS read count of a nanobody compared to the total read count of all nanobodies in Round 3 output, fold change of a nanobody's abundance from Round 1 input to Round 1 output, fold change of a nanobody's abundance from Round 2 input to Round 2 output, fold change of a nanobody's abundance from Round 3 input to Round 3 output, rotal number of rounds where a nanobody enriched (enrichment>1), growth bias between Round 1 and 2 (Fold change of a nanobody's abundance from Round 1 output to Round 2 input), growth bias between Round 2 and 3 (Fold change of a nanobody's abundance from Round 2 output to Round 3 input), mean fluorescent intensity of FLAG staining on yeast as measured by flow cytometry, percentage of yeast stained positively by FLAG as measured by flow cytometry, and mean fluorescent intensity of polyclonal phage binding on yeast after Round 3, as measured by flow cytometry.

In some embodiments, a nanobody is identified as having high affinity for its target when the binding score is at least 0.5, at least 0.6, at least 0.7, at least 0.8, at least 0.9, at least 0.91, at least 0.92, at least 0.93, at least 0.94, at least 0.95, at least 0.96, at least 0.97, at least 0.98, at least 0.99 or greater than 0.99.

### Methods of Using the Binding Molecules

Given the properties of the secreted or extracellular protein binding molecules (e.g., nanobodies, etc.) of the present invention, the secreted or extracellular protein binding molecules are suitable as diagnostic, therapeutic and prophylactic agents for diagnosing, treating or preventing diseases, disorders and associated conditions in humans and animals.

In general, use comprises administering a therapeutically or prophylactically effective amount of one or more nanobodies or binding fragments of the present invention to a susceptible subject or one exhibiting a condition in which the target activity is known to have pathological sequelae. Any active form of the secreted or extracellular protein binding molecules (e.g., nanobodies, etc.) can be administered, including antibody Fab and F(ab′)2 fragments.

In some embodiments, the secreted or extracellular protein binding molecule used is compatible with the recipient species such that an immune response to the secreted or extracellular protein binding molecule does not result in an unacceptably short circulating half-life or induce an immune response to the secreted or extracellular protein binding molecule in the subject.

Treatment of individuals may comprise the administration of a therapeutically effective amount of the secreted or extracellular protein binding molecule of the present invention. The secreted or extracellular protein binding molecule can be provided in a kit as described below. The secreted or extracellular protein binding molecule can be used or administered as a mixture, for example in equal amounts, or individually, provided in sequence, or administered all at once. In providing a patient with the secreted or extracellular protein binding molecule, the dosage of administered agent will vary depending upon such factors as the patient's age, weight, height, sex, general medical condition, previous medical history, etc.

In general, if administering a systemic dose of a secreted or extracellular protein binding molecule, it is desirable to provide the recipient with a dosage of a secreted or extracellular protein binding molecule which is in the range of from about 1 ng/kg-100 ng/kg, 100 ng/kg-500 ng/kg, 500 ng/kg-1 μg/kg, 1μg/kg-100 μg/kg, 100 μg/kg-500 μg/kg, 500 μg/kg-1 mg/kg, 1 mg/kg-50 mg/kg, 50 mg/kg-100 mg/kg, 100 mg/kg-500 mg/kg (body weight of recipient), although a lower or higher dosage may be administered. Dosages as low as about 1.0 mg/kg may be expected to show some efficacy. Preferably, about 5 mg/kg is an acceptable dosage, although dosage levels up to about 50 mg/kg are also preferred especially for therapeutic use. Alternatively, administration of a specific amount of the secreted or extracellular protein binding molecule may be given which is not based upon the weight of the patient such as an amount in the range of 1 μg-100 μg, 1 mg-100 mg, or 1 gm-100 gm. For example, site specific administration may be to body compartment or cavity such as intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, vaginal, rectal, buccal, sublingual, intranasal, ophthalmic,or transdermal means.

The secreted or extracellular protein binding molecule composition can be prepared for use for parenteral (subcutaneous, intramuscular or intravenous) or any other administration particularly in the form of liquid solutions or suspensions; for use in vaginal or rectal administration particularly in semisolid forms such as, but not limited to, creams and suppositories; for buccal, or sublingual administration such as, but not limited to, in the form of tablets or capsules; or intranasally such as, but not limited to, the form of powders, nasal drops or aerosols or certain agents; or ophthalmically such as, but not limited to, eye drops; or for the treatment of dental disease; or transdermally such as not limited to a gel, ointment, lotion, suspension or patch delivery system with chemical enhancers such as dimethyl sulfoxide to either modify the skin structure or to increase the drug concentration in the transdermal patch, or with oxidizing agents that enable the application of formulations containing proteins and peptides onto the skin (WO 98/53847), or applications of electric fields to create transient transport pathways such as electroporation, or to increase the mobility of charged drugs through the skin such as iontophoresis, or application of ultrasound such as sonophoresis (U.S. Pat. Nos. 4,309,989 and 4,767,402).

The secreted or extracellular protein binding molecules of the present invention can be formulated according to known methods to prepare pharmaceutically useful compositions, whereby these materials, or their functional derivatives, are combined in admixture with a pharmaceutically acceptable carrier vehicle. Suitable vehicles and their formulation, inclusive of other human proteins, e.g., human serum albumin, are described, for example, in Remington's Pharmaceutical Sciences (16th ed., Osol, A. ed., Mack Easton Pa. (1980)). In order to form a pharmaceutically acceptable composition suitable for effective administration, such compositions will contain an effective amount of the above-described compounds together with a suitable amount of carrier vehicle. Additional pharmaceutical methods may be employed to control the duration of action. Controlled release preparations may be achieved through the use of polymers to complex or absorb the compounds. Another possible method to control the duration of action by controlled release preparations is to incorporate the compounds of the present invention into particles of a polymeric material such as polyesters, polyamino acids, hydrogels, poly(lacticacid) or ethylene vinylacetate copolymers. Alternatively, instead of incorporating these agents into polymeric particles, it is possible to entrap these materials in microcapsules prepared, for example, interfacial polymerization, for example, hydroxymethylcellulose or gelatin-microcapsules and poly(methylmethacylate)-microcapsules, respectively, or in colloidal drug delivery systems, for example, liposomes, albumin microspheres, microemulsions, nanoparticles, and nanocapsules or in macroemulsions.

The treatment may be given in a single dose schedule, or preferably a multiple dose schedule in which a primary course of treatment may be with 1-10 separate doses, followed by other doses given at subsequent time intervals required to maintain and or reinforce the response, for example, at 1-4 months for a second dose, and if needed, a subsequent dose(s) after several months. Examples of suitable treatment schedules include: (i) 0, 1 month and 6 months, (ii) 0, 7 days and 1 month, (iii) 0 and 1 month, (iv) 0 and 6 months, or other schedules sufficient to elicit the desired responses expected to reduce disease symptoms, or reduce severity of disease.

### Methods of Diagnosis

In some embodiments, an increase in the level of a secreted or extracellular protein, in a subject's cell, tissue, or bodily fluid, compared with a comparator is used in the methods of the invention as marker for the diagnosis of a disease or disorder, assessing the severity of a disease or disorder, and for monitoring the effect or effectiveness of a treatment of a disease or disorder.

In one embodiment, the invention is a method of diagnosing a disease or disorder by assessing the level of a secreted or extracellular protein in a patient. Non-limiting examples of this embodiment would be the use of a binder against a secreted or extracellular protein for PET imaging, SPECT imaging, MRI imaging, radiographic imaging, ultrasound imaging, fluorescence imaging, or bioluminescent imaging. In such examples, the binder that recognizes an extracellular or secreted protein may be fused or conjugated to a detection moiety. These moieties may include (but are not limited to) a radioisotope, a magnetic spin-label, a fluorophore, a fluorescent protein, or a bioluminescent protein.

In one embodiment, the invention is a method of diagnosing a disease or disorder of a subject by assessing the level of a secreted or extracellular protein, in a biological sample of the subject. In one embodiment, the biological sample of the subject is a cell, tissue, or bodily fluid. Non-limiting examples of bodily fluids in which the level of a secreted or extracellular protein, can be assessed include, but are not limited to, blood, serum, plasma and urine. In various embodiments, the level of a secreted or extracellular protein, in the biological sample of the subject is compared with the secreted or extracellular protein level in a comparator. Non-limiting examples of comparators include, but are not limited to, a negative control, a positive control, an expected normal background value of the subject, a historical normal background value of the subject, an expected normal background value of a population that the subject is a member of, or a historical normal background value of a population that the subject is a member of In some embodiments, the method of diagnosing includes a further step of treating the patient for the diagnosed disease or disorder.

In another embodiment, the invention is a method of assessing the severity of a disease or disorder of a subject by assessing the level of a secreted or extracellular protein in a biological sample of the subject. In one embodiment, the biological sample of the subject is a cell, tissue, or bodily fluid. Non-limiting examples of bodily fluids in which the level of a secreted or extracellular protein can be assessed include, but are not limited to, blood, serum, plasma and urine. In various embodiments, the level of a secreted or extracellular protein in the biological sample of the subject is compared with the secreted or extracellular protein level in a comparator. Non-limiting examples of comparators include, but are not limited to, a negative control, a positive control, an expected normal background value of the subject, a historical normal background value of the subject, an expected normal background value of a population that the subject is a member of, or a historical normal background value of a population that the subject is a member of In some embodiments, the method of assessing the severity includes a further step of treating the patient for the disease or disorder.

In another embodiment, the invention is a method of monitoring the effect of a treatment of a disease or disorder of a subject by assessing the level of a secreted or extracellular protein in a biological sample of the subject. In one embodiment, the biological sample of the subject is a cell, tissue, or bodily fluid. Non-limiting examples of bodily fluids in which the level of a secreted or extracellular protein can be assessed include, but are not limited to, blood, serum, plasma and urine. In various embodiments, the level of a secreted or extracellular protein in the biological sample of the subject is compared with the secreted or extracellular protein level in a comparator. Non-limiting examples of comparators include, but are not limited to, a negative control, a positive control, an expected normal background value of the subject, a historical normal background value of the subject, an expected normal background value of a population that the subject is a member of, or a historical normal background value of a population that the subject is a member of In some embodiments, the method of monitoring the effect of a treatment includes a further step of treating the patient for the disease or disorder.

In various embodiments, the subject is a human subject, and may be of any race, sex and age. Representative subjects include those who are suspected of having experienced a disease or disorder, those who have been diagnosed as having experienced a disease or disorder, those who have been diagnosed as having a disease or disorder, and those who are at risk of developing a disease or disorder.

Information obtained from the methods of the invention described herein can be used alone, or in combination with other information (e.g., disease status, disease history, vital signs, blood chemistry, etc.) from the subject or from the biological sample obtained from the subject.

In the diagnostic methods of the invention, a biological sample obtained from a subject is assessed for the level of a secreted or extracellular protein contained therein. In one embodiment, the biological sample is a sample containing at least a fragment of a secreted or extracellular protein useful in the methods described herein.

In other various embodiments of the methods of the invention, the level of a secreted or extracellular protein is determined to be increased when the level of a secreted or extracellular protein is increased by at least 10%, by at least 20%, by at least 30%, by at least 40%, by at least 50%, by at least 60%, by at least 70%, by at least 80%, by at least 90%, by at least 100%, by at least 200%, by at least 300%, by at least 400%, by at least 500%, by at least 600%, by at least 700%, by at least 800%, by at least 900%, by at least 1000%, when compared to with a comparator control. In various embodiments, an increased level of a secreted or extracellular protein is indicative of a disease or disorder.

In the methods of the invention, a biological sample from a subject is assessed for the level of a secreted or extracellular protein in the biological sample obtained from the patient. The level of a secreted or extracellular protein in the biological sample can be determined by assessing the amount of target polypeptide, or a fragment thereof, in the biological sample, the amount of target mRNA, or a fragment, in the biological sample, the amount of target activity (e.g., enzymatic activity, substrate binding activity, receptor binding activity, etc.) in the biological sample, or a combination thereof. In some embodiments, the level of a secreted or extracellular protein in the biological sample is determined in an assay using at least one of the secreted or extracellular protein binding molecules of the invention described elsewhere herein.

Methods of measuring a secreted or extracellular protein levels in a biological sample obtained from a patient include, but are not limited to, an immunochromatography assay, an immunodot assay, a Luminex assay, an ELISA assay, an ELISPOT assay, a protein microarray assay, a Western blot assay, a mass spectrophotometry assay, a radioimmunoassay (MA), a radioimmunodiffusion assay, a liquid chromatography-tandem mass spectrometry assay, an ouchterlony immunodiffusion assay, reverse phase protein microarray, a rocket immunoelectrophoresis assay, an immunohistostaining assay, an immunofluorescence assay, an immunoprecipitation assay, a complement fixation assay, FACS, flow cytometry, mass cytometry (e.g., CyTOF), CITE-seq, REAP-seq, MIBI-TOF, an enzyme-substrate binding assay, an enzymatic assay, an enzymatic assay employing a detectable molecule, such as a chromophore, fluorophore, or radioactive substrate, a isotopic label, a substrate binding assay employing such a substrate, a substrate displacement assay employing such a substrate, and a protein chip assay (see also, 2007, Van Emon, Immunoassay and Other Bioanalytical Techniques, CRC Press; 2005, Wild, Immunoassay Handbook, Gulf Professional Publishing; 1996, Diamandis and Christopoulos, Immunoassay, Academic Press; 2005, Joos, Microarrays in Clinical Diagnosis, Humana Press; 2005, Hamdan and Righetti, Proteomics Today, John Wiley and Sons; 2007). In some embodiments, the level of a secreted or extracellular protein in the biological sample is measure with an assay that uses at least one of the secreted or extracellular protein binding molecules of the invention that are described elsewhere herein.

### Kits

The present invention also provides kits which are useful for carrying out the present invention. In some embodiments, the kit comprises one or more secreted or extracellular protein binding molecule which binds an epitope of its target protein as provided in Table 2 of the invention and an instructional material which describes, for instance, administering the secreted or extracellular protein binding molecule to an individual as a therapeutic treatment or use of the secreted or extracellular protein binding molecule in an assay as described elsewhere herein.

The present kits comprise a first container containing or packaged in association with the above-described nanobodies. The kit may also comprise another container containing or packaged in association solutions necessary or convenient for carrying out the invention. The containers can be made of glass, plastic or foil and can be a vial, bottle, pouch, tube, bag, etc. The kit may also contain written information, such as procedures for carrying out the present invention or analytical information, such as the amount of reagent contained in the first container means. The container may be in another container apparatus, e.g. a box or a bag, along with the written information.

Yet another aspect of the present invention is a kit for detecting a secreted or extracellular protein in a biological sample. In some embodiments, the kit includes a container holding one or more secreted or extracellular protein binding molecule which binds an epitope of its target protein as provided in Table 2 and instructions for using the secreted or extracellular protein binding molecule for the purpose of binding to the target to form complex and detecting the formation of the complex such that the presence or absence of the complex correlates with presence or absence of the target in the sample. Examples of containers include multi-well plates which allow simultaneous detection of the target in multiple samples.

In some embodiments, the kit further comprises a carrier suitable for dissolving or suspending the secreted or extracellular protein binding molecule, or combinations thereof, of the invention, for instance, a pharmaceutically acceptable carrier for dissolving or suspending the secreted or extracellular protein binding molecule prior to administering the secreted or extracellular protein binding molecule of the invention to an individual. Optionally, the kit comprises an applicator for administering the secreted or extracellular protein binding molecule.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Display of Human Antigens on the Surface of Yeast

### Library Design

An initial library of 1384 human extracellular proteins was assembled based on protein domains, immunological functions, and yeast-display compatibility. The extracellular portion of each protein was identified by manual inspection of topological domains annotated in the SwissProt database (January 2018). For proteins with uncertain topology, full sequences were run through SignalP 4, Topcons, and GPIPred to identify most likely topologies. For proteins with multiple extracellular portions, in general the longest individual region was chosen for initial amplification. cDNAs for chosen proteins were purchased from GE Dharmacon or DNASU. The protein sequences were further modified to match isoforms available in purchased cDNAs. Proteins whose cDNAs were not purchased were ordered as synthesized gene fragments produced by Twist Bioscience. This human exoproteome library was expanded to encompass an additional 1704 human extracellular proteins for a total of 3088, representing the set of 59.9% of extracellular proteins and nearly all proteins with ectodomain/secreted regions>50 amino acids.

### Library Construction

A two-step PCR process was used to amplify cDNAs for cloning into a barcoded yeast-display vector. cDNAs were amplified with gene-specific primers, with the forward primer containing a 5′ sequence (CTGTTATTGCTAGCGTTTTAGCA; SEQ ID NO: 42897) and the reverse primer containing a 5′ sequence (GCCACCAGAAGCGGCCGC; SEQ ID NO:43898) for template addition in the second step of PCR. PCR reactions were conducted using 1 μL pooled cDNA, gene-specific primers, and the following PCR settings: 98° C. denaturation, 58° C. annealing, 72° C. extension, 35 rounds of amplification. 1μL of PCR product was used for direct amplification by common primers Aga2FOR and 159REV, and the following PCR settings: 98° C. denaturation, 58° C. annealing, 72° C. extension, 35 rounds of amplification. PCR product was purified using magnetic PCR purification beads (AvanBio). 90 μL beads were added to the PCR product and supernatant was removed. Beads were washed twice with 200 μL 70% ethanol and resuspended in 50 μL water to elute PCR products from the beads. Beads were removed from purified PCR products. The 15 bp barcode fragment was constructed by overlap PCR. 4 primers (bc1, bc2, bc3, bc4) were mixed in equimolar ratios and used as template for a PCR reaction using the following PCR settings: 98° C. denaturation, 55° C. annealing, 72° C. extension, 35 rounds of amplification. Purified product was reamplified with the first and fourth primer using identical PCR conditions. PCR products were run on 2% agarose gels and purified by gel extraction (Qiagen). Purified barcode and gene products were combined with linearized yeast-display vector (pDD003 digested with EcoRI and BamHI) and electroporated into JAR300 yeast cell using a 96-well electroporater (BTX Harvard Apparatus) using the following electroporation conditions: Square wave, 500 V, 5 ms pulse, 2 mm gap. Yeast cell were immediately recovered into 1 mL liquid synthetic dextrose medium lacking uracil (SDO-Ura) in 96-well deepwell blocks and grown overnight at 30° C. Yeast cell were passaged once by 1:10 dilution in SDO-Ura, then frozen as glycerol stocks. To induce antigen expression, yeast were recovered and expanded in liquid synthetic dextrose medium with casamino acids (SDCAA) at 30° C. for 24 hours, and induced by diluting 1:10 into liquid synthetic galactose medium with casamino acids (SGCAA) and cultured at 30° C. for 24 hours. 10⁷ yeast cells were pelleted for each well in V bottom 96-well plates (BrandTech) and resuspend in 150 μL PBE (PBS with 0.5% BSA and 0.5 mM EDTA) containing 1:500 biotinylated anti-FLAG antibody (GenScript). Yeast cell were stained at 4° C. for 1 hour, and washed three times with 200 μL PBE. FLAG displaying yeast was magnetically enriched using Streptavidin coated magnetic beads 0.24-0.47 um, 0.5% w/v (SPHERO). Yeast were expanded in liquid synthetic dextrose medium with casamino acids (SDCAA) at 30° C. overnight, then frozen as glycerol stocks. To induce antigen expression, FLAG enriched yeast were recovered and expanded in liquid synthetic dextrose medium with casamino acids (SDCAA) at 30° C. for 24 hours, and induced by diluting 1:10 into liquid synthetic galactose medium with casamino acids (SGCAA) and cultured at 30° C. for 24 hours. Display of antigens was quantitated by incubating yeast cells in PBE containing 1:3,000 anti-FLAG PE antibody (BioLegend) at 4° C. for 30 minutes and detecting FLAG display on a flow cytometer (SONY).

### Example 2: Display of a Synthetic Nanobody Library on the Surface of Bacteriophage

### Phage Displayed Nanobody Library Assembly

A synthetic nanobody library was displayed on the surface of M13 filamentous bacteriophage. The synthetic nanobody library was constructed as described before (McMahon, C. et al, Nat Struct Mol Biol. 2018). The nanobody library fragments were digested with restriction enzymes FseI and NsiI at 37° C. for 1 hour and purified by gel extraction (Qiagen). A P3 gene surface display phagemid vector p443 were subjected to restriction digestion at FseI and NsiI at 37° C. for 1 hour and purified by gel extraction (Qiagen). Purified nanobody library fragments and linearized p443 vector were combined and ligated with T4 DNA Ligase (NEB) at 16° C. overnight. Ligation product was purified by Qiaquick PCR Purification Kit (Qiagen) and eluted in water. The purified phagemid library was electroporated into electrocompetent E. coli strain SR320 with the following electroporation settings: 2500 V, 125Ω, and 50 μF. E. coli cells were immediately recovered into 5 mL S.O.C. medium (Corning) for 1 hour by shaking at 37° C. at rotation speed of 220 rpm. M13K07 helper phage particles were added to a final concentration of 10¹⁰ phage per mL for 1 hour at 37° C. The mixture was expanded in 1 L 2YT medium supplemented with Tetracycline (at final concentration of 10 μg/mL), Kanamycin (at final concentration of 50 μg/mL) and Carbenicillin (at final concentration of 100 μg/mL) overnight at 37° C. 10 μL of fresh electroporation culture was serially diluted and plated on LB/Carbenicillin agar plates overnight to estimate the number of colony forming units in the electroporation mixture. The overnight culture was centrifuged, and the phage particles were harvested from the supernatant by precipitation with ⅕ final volume of PEG/NaCl on ice for 30 minutes. The precipitate was resuspended in PBS. A final concentration of 10% glycerol was added and thoroughly mixed to the phage solution before aliquoting and storage at −80° C. The resulting library contained 2.4×10¹⁰ transformants. Each aliquot contained 10¹³ phage particles.

### Selection of HA Displaying Phage Nanobody Library

The naïve library was selected for expression of HA tag and counter-selected against binding for Neutravidin or promiscuous binding to plates. Anti-HA antibody and Neutravidin (Thermo Fischer Scientific) was immobilized on 96-well MaxiSorp plate (Thermo Scientific) at 4° C. overnight, at a final concentration of 25 pg/mL in PBS. The plates was blocked in Blocking Buffer (PBS with 0.5% BSA) for 2 hours at room temperature. The naïve library was applied onto the Neutravidin plate and incubated at room temperature for 1 hour. Collect the unbound phage solution from Neutravidin plate and apply onto anti-HA plate. The anti-HA plate was incubated at room temperature for 2 hours. The plate was washed 6 times with PT (PBS with 0.05% Tween 20). The phage particles were eluted by incubation with 0.1N HCl solution at room temperature for 5-10 minutes. The elution solution was collected and neutralized by addition of ⅛ volume of Tris pH 11 solution. The mixture was added to 50 mL of log-phase OmniMax culture and cultured for 45 minutes at 37° C. with 220 rpm shaking. M13K07 helper phage particles were added to a final concentration of 10¹⁰ phage per mL for 1 hour at 37° C. The culture was expanded to 1 L of 2YT medium supplemented with Kanamycin and Carbenicillin, and cultured overnight. The overnight culture was centrifuged, and the phage particles were harvested from the supernatant by precipitation with ⅕ final volume of PEG/NaCl on ice for 30 minutes. The precipitate was resuspended in PBS. A final concentration of 10% glycerol was added and thoroughly mixed to the phage solution before aliquoting and storage at −80° C. Each aliquot contained 10¹³ phage particles.

### Example 3: High-Throughput Selection of Phage Displayed Nanobodies on Yeast Displayed Antigens

### HAPPY Selection Procedures

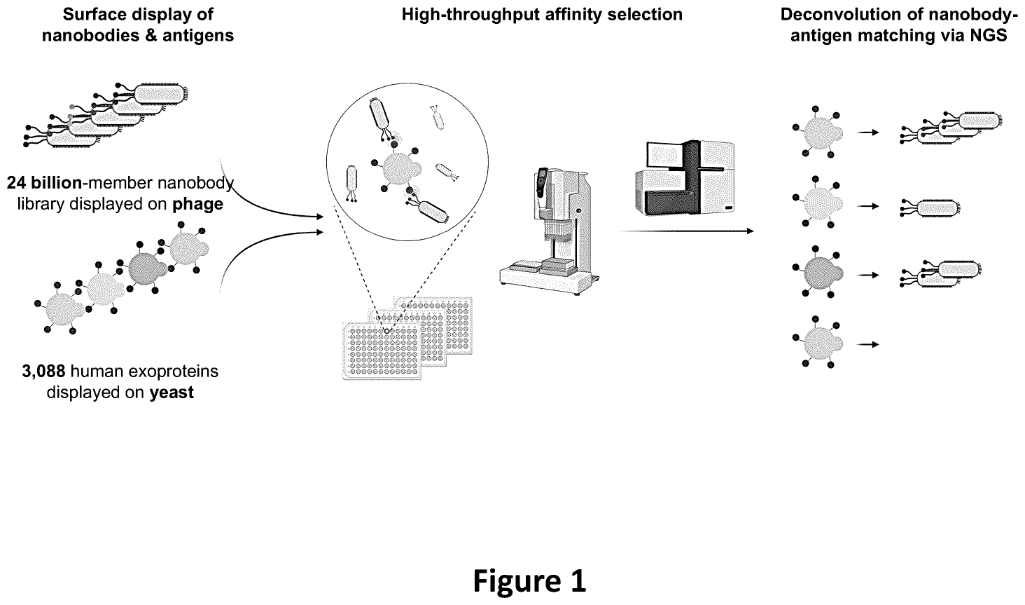

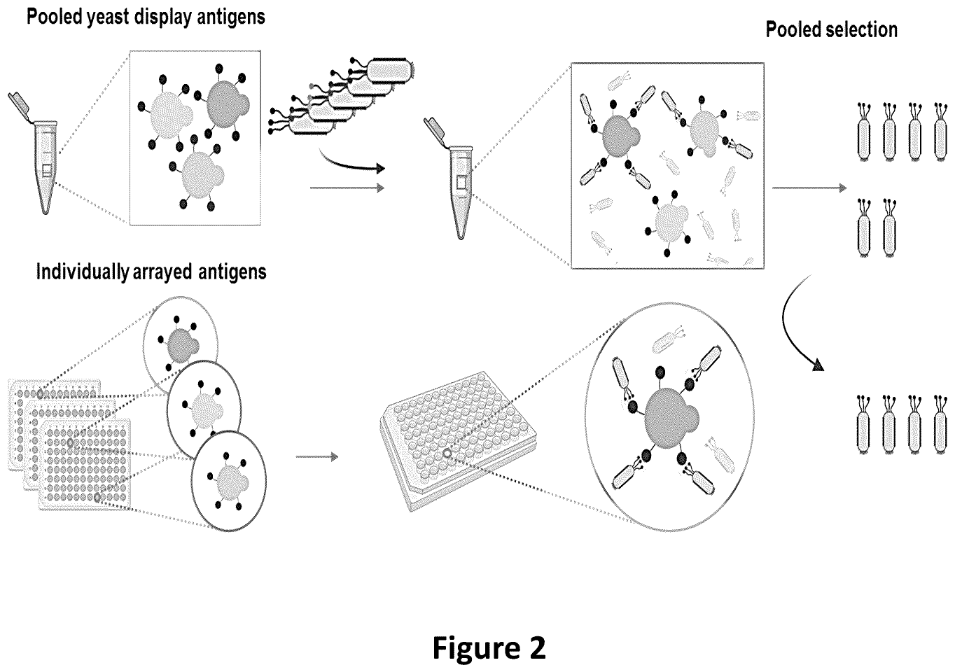

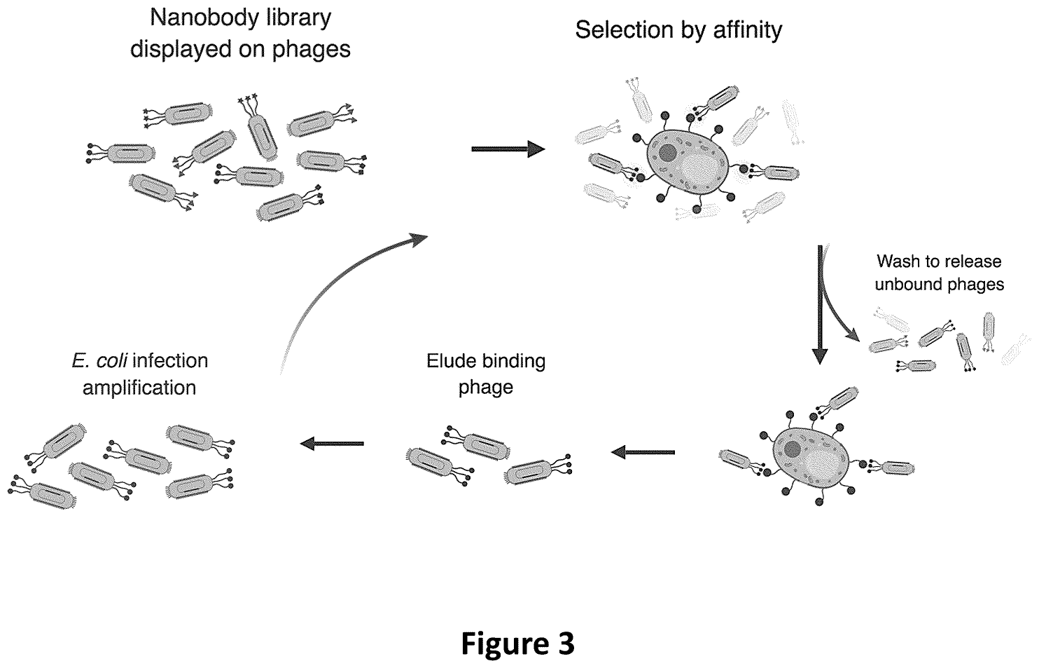

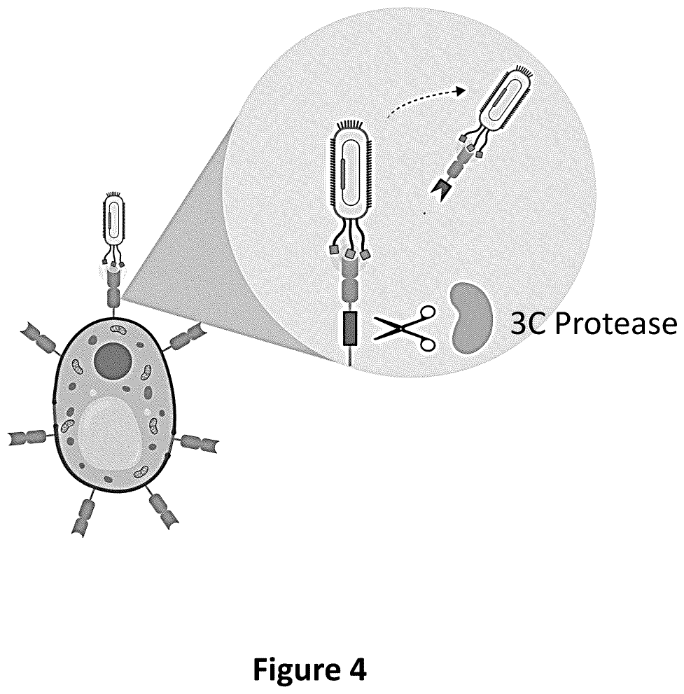

A comprehensive nanobody selection campaign was performed on 3,088 human extracellular and secreted proteins (FIG. 1). The strategy used to select phage nanobodies specific to yeast antigens is shown in FIG. 2. This workflow, termed HAPPY (High Throughput Antibody discovery by Panning Phage on Yeast), enabled high-throughput affinity selection. In brief, the high-throughput selection pipeline entailed incubation of the collection of nanobody-phage library with a mixed pool of yeast, containing 96 unique strains at a time. Unbound phage were removed by washing the yeast, and the mixed pool of reactive phage were eluted from the surface of yeast. The phage output was amplified, purified, and subjected to a second round of selection of the same process (FIG. 3). The elution was achieved via a proteolytic reaction with HRV 3C protease that enables specific release of phage from displayed antigens (FIG. 4). Lastly, the selection output from the second round were subjected to a third round of selection comprised of individual selections on single yeast strains displaying a unique protein antigen in a highly parallelized process in microtiter plates. After the third round of selection, enriched nanobodies for each antigen were identified by sequencing the phage genomic DNA via next generation sequencing.

To induce antigen expression, yeast were recovered and expanded in liquid synthetic dextrose medium with casamino acids (SDCAA) at 30° C., and induced by diluting 1:10 into liquid synthetic galactose medium with casamino acids (SGCAA) and cultured at 30° C. for 24 hours. All selection steps were carried out at 30° C. using PBE buffer (PBS with 0.5% BSA and 2 mM EDTA). For Round 1, approximately 10¹³ phage particles were counter-selected with 10⁹ yeast cells transformed with pDD003 expressing only the empty vector. Positive selection was performed by applying the counter-selected phage pool to a mixed pool of 96 antigen displaying yeasts, with approximately 10⁷ yeast cells for each antigen. After washing, 3C protease was added to release antigen binding phage. The polyclonal phage output was amplified via infection of E. coli OmniMax2 (ThermoFischer). The infection culture was purified and subjected to the second round of selection. On the third round of selection, the counter-selection was performed as the previous rounds. The counter-selected phage were split into 96-well plates, each well containing one strain of yeast. Each well was individually washed and eluted.

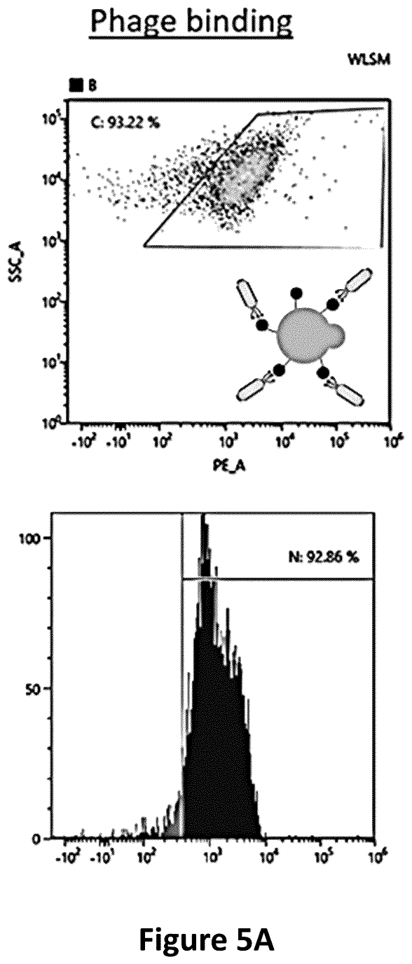

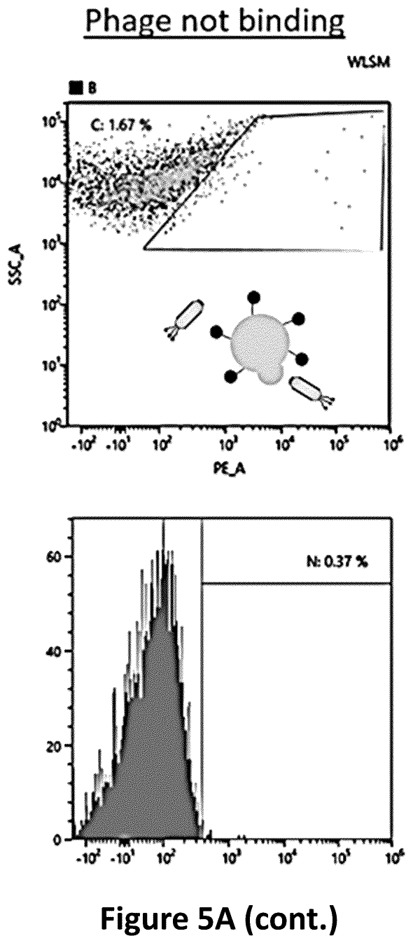

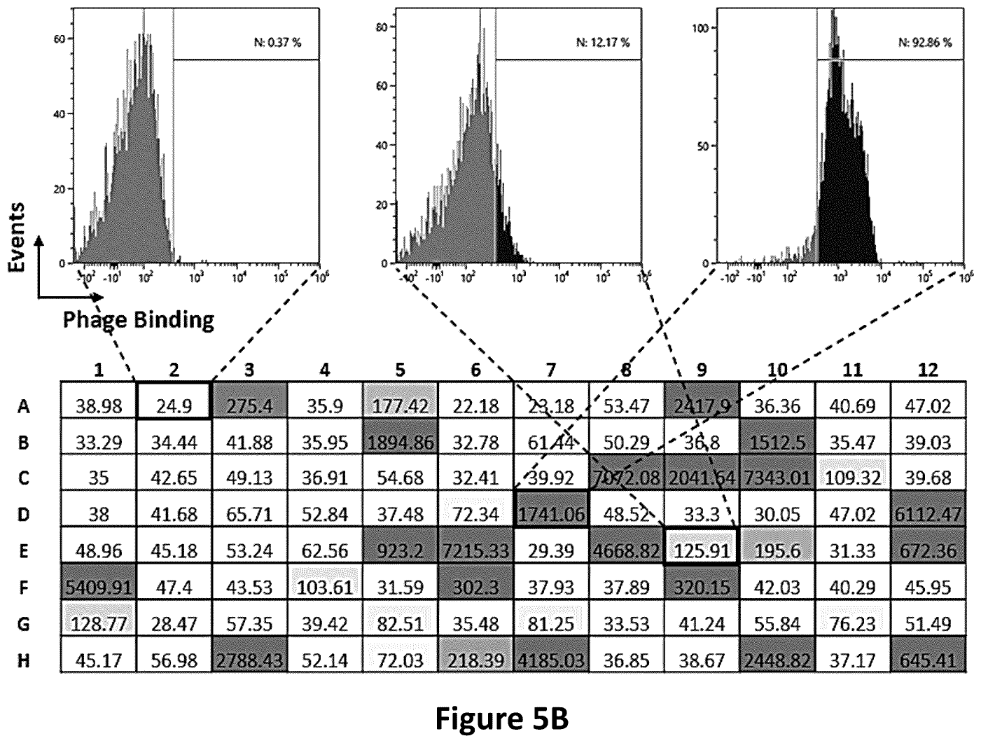

The general enrichment of reactive phage in the polyclonal phage pool of each well can be monitored by applying the polyclonal phage output to the antigen yeast, and detecting phage binding on yeast by flow cytometry using anti-HA PE antibodies (BioLegend) (FIG. 5).

### Next Generation Sequencing Library Preparation and Sequencing

To sequence the nanobodies, a two-step PCR process was employed to prepare next generation sequencing library. A first round of PCR was used to amplify a DNA sequence containing the nanobody region on the phagemid. The overnight amplification culture of polyclonal phage output form the final round of selection was used to as the template. PCR reactions were conducted using primers FL3(GTCTCGTGGGCTCGGAGATGTGTATAAGAGACAGNNNNNNNNCGTATGGGTAAGAACCACCGCT; SEQ ID NO:42899) and FL9(TCGTCGGCAGCGTCAGATGTGTATAAGAGACAGNNNNNNNNAGCTGCGCGGCGAGC; SEQ ID NO:42900). PCR product was purified using magnetic PCR purification beads (AvanBio). 45 μL beads were added to the PCR product and supernatant was removed. Beads were washed twice with 100 μL 70% ethanol and resuspended in 25 μL water to elute PCR products from the beads. Beads were removed from purified PCR products. A second round of PCR was conducted using 1 μL purified PCR product and Nextera i5 and i7 dual-index library primers (Illumina). The concentration of outer PCR product of each well of a plate was normalized using Just-a-Plate (Charm Biotech). The normalized product was pooled and concentrated using a Qiagen DNA purification column (Qiagen). The concentrated product was run on a 2% agarose gel, and a band at approximately 450 base pairs was excised and extracted using a QlAquicl Gel Extraction Kit (Qiagen) according to standard manufacturer protocols. The DNA library was sequenced with Illumina Hi Seq and Illumina v2 300 PE kit according to standard manufacturer protocols.

### Example 4: Nanobody Expression, Purification, and Characterization

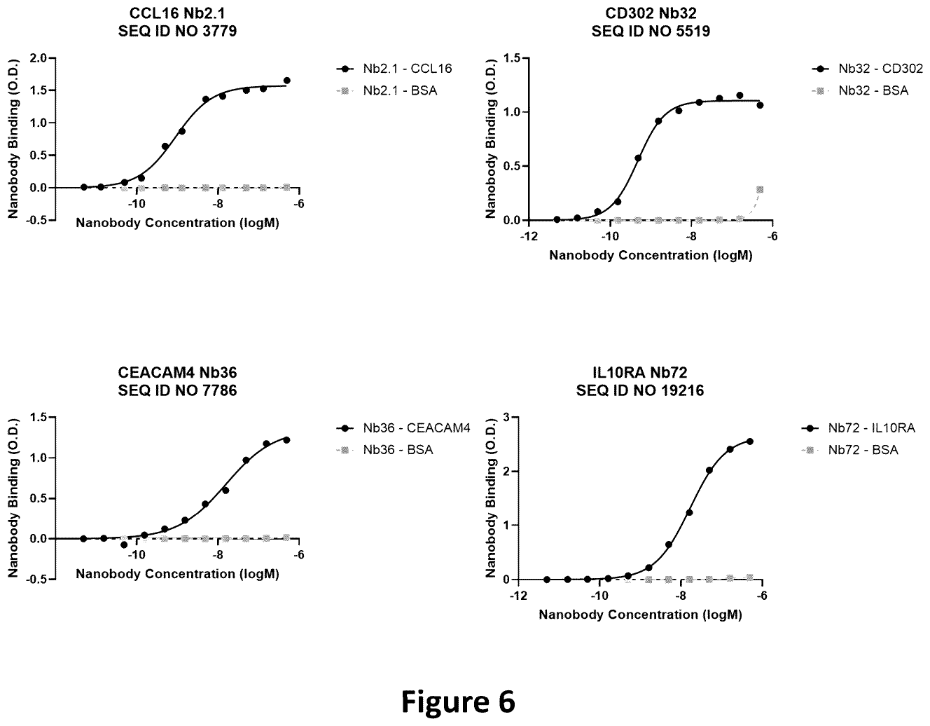

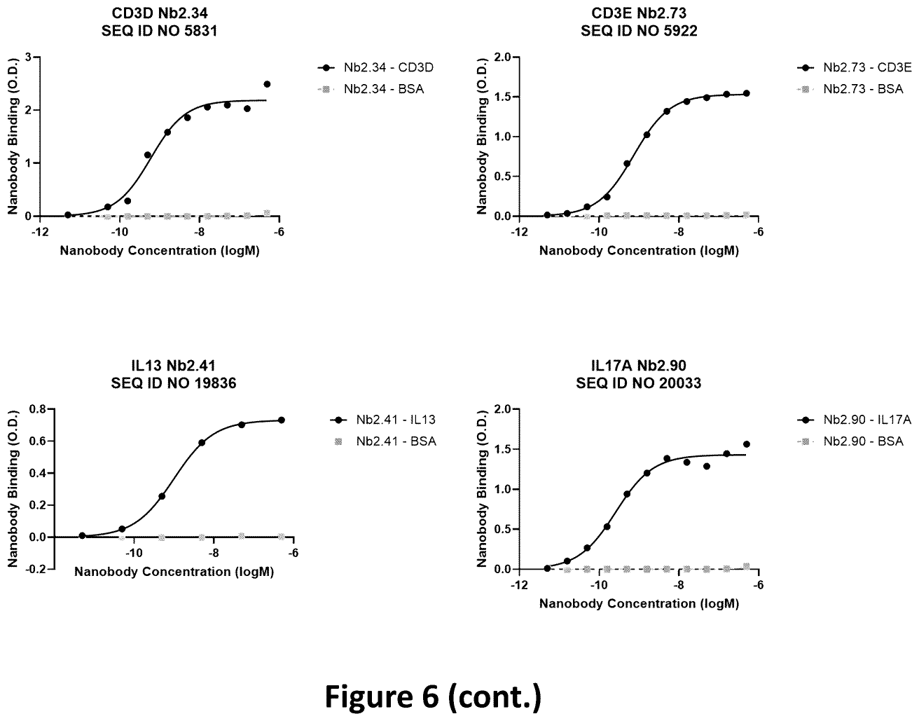

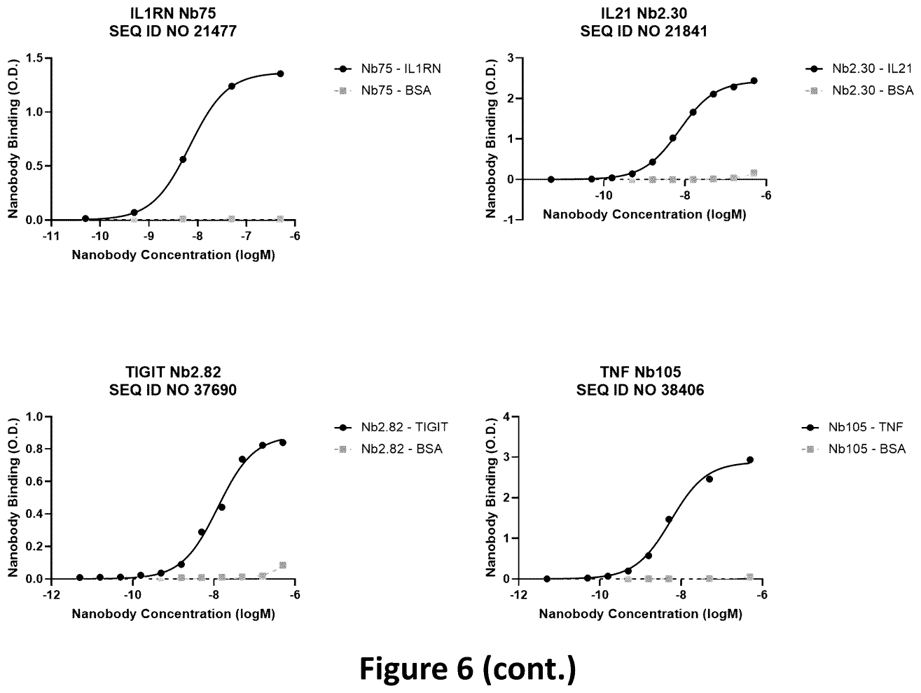

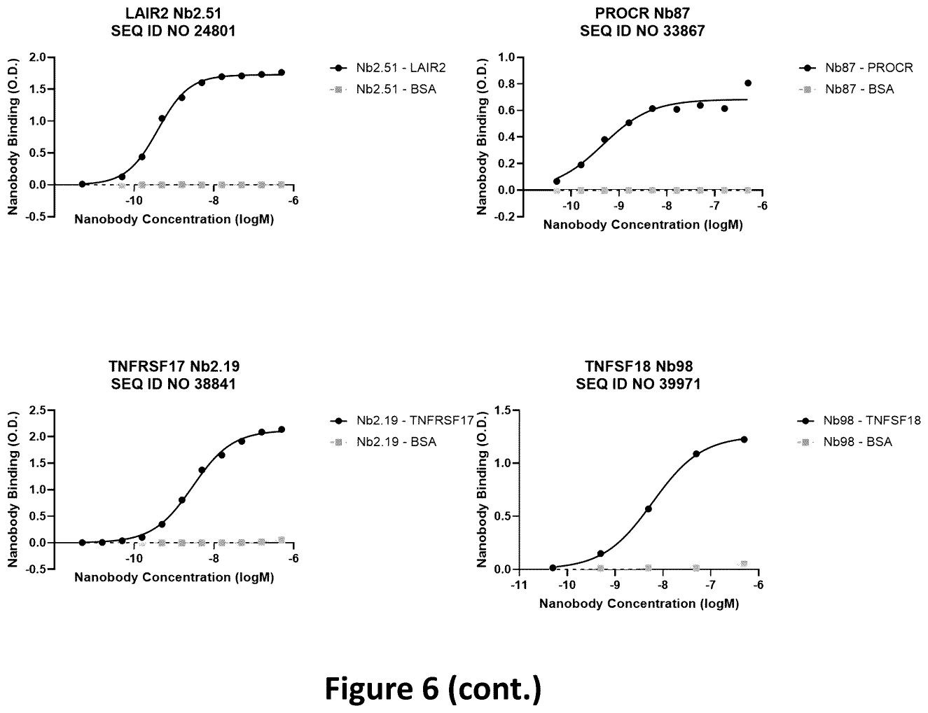

Individual nanobodies were recombinantly expressed and characterized by ELISA and Biolayer Interferometry. Examples were shown in FIG. 6. Additional validation data was included in Table 1.

### Nanobody Protein Production

The nanobodies were expressed as a Fc fusion protein by cloning into a mammalian expression vector pCER243. Protein was produced by transfection into Expi293 cells (Thermo Fisher Scientific) according to standard manufacturer protocols. Transfected cells were maintained according to manufacturer protocols. 4 days post-transfection, media was clarified by centrifugation at 300 g for 5 minutes. Protein was purified from clarified media using Protein A magnetic beads (Lytic Solutions). Protein purity was verified by SDS-PAGE.

### ELISAs

Recombinant human protein was diluted in PBS to a final concentration of 5 μg/mL and was immobilized on 96-well flat bottom MaxiSorp plates (Thermo Fisher Scientific) and placed at 4° C. overnight. Plates were washed three times with 225 μL ELISA wash buffer (PBS with 0.05% Tween 20) and 200 μL ELISA blocking buffer (PBS with 1% Casein) was added to the well. Plates were incubated with shaking for 2 hours at room temperature. ELISA blocking buffer was removed from the wells and appropriate dilutions of recombinant nanobodies in 100 μL ELISA blocking buffer was added to each well. Plates were incubated with shaking for 2 hours at room temperature. Plates were washed 3 times with 225 μL ELISA wash buffer and 1:5000 goat anti-human IgG HRP (Millipore Sigma) in 100 μL ELISA blocking buffer was added to the wells. Plates were incubated with shaking for 1 hour at room temperature. Plates were washed 6 times with 225 μL ELISA wash buffer. 50 μL TMB substrate (BD Biosciences) was added to the wells and plates were incubated for 15 minutes in the dark at room temperature. 50 μL 1 M sulfuric acid was added to the wells and absorbance at 450 nm was measured in a Synergy HTX Multi-Mode Microplate Reader (BioTek). FIG. 6 provides representative examples of ELISA assay results that demonstrate the specific, does-dependent binding of the nanobodies to the antigens.

### Biolayer Interferometry Assays

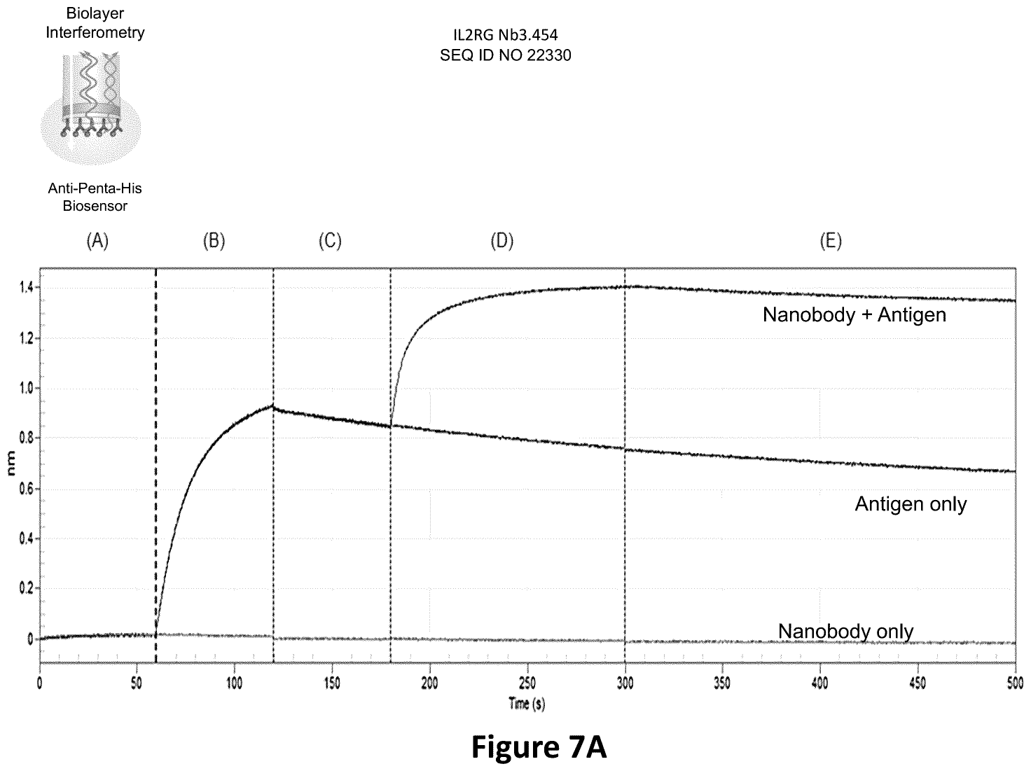

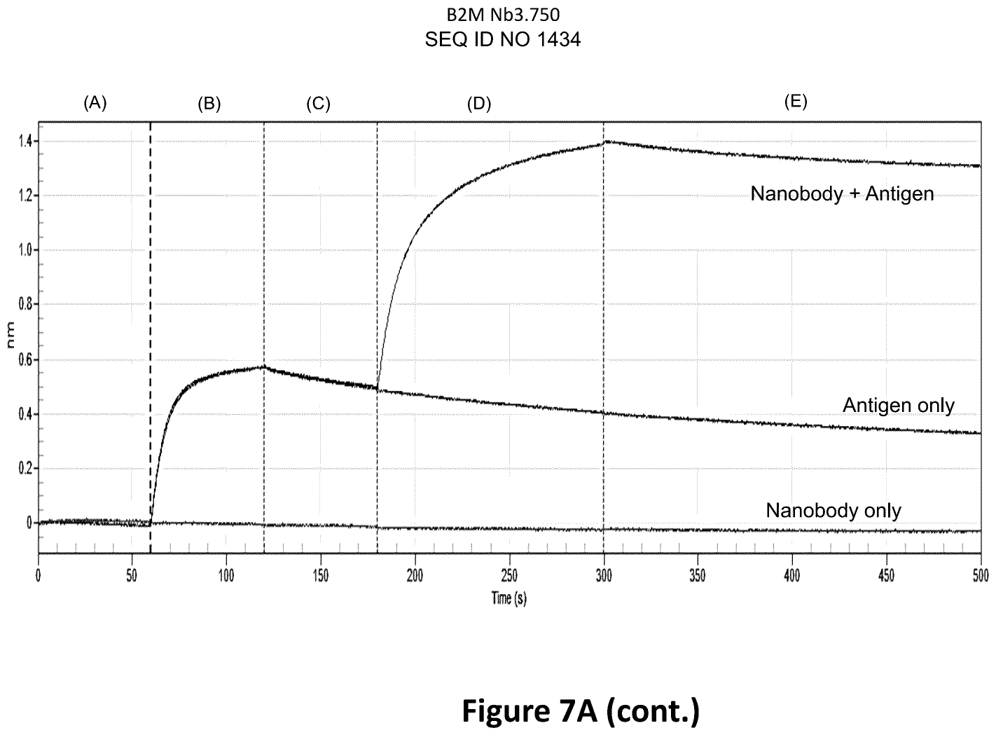

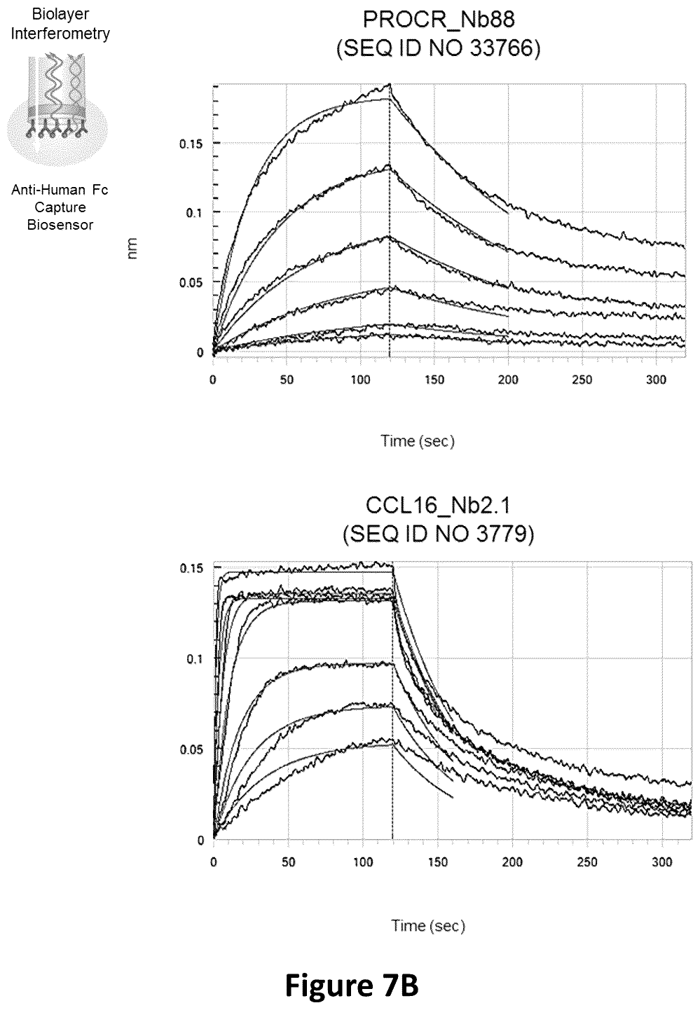

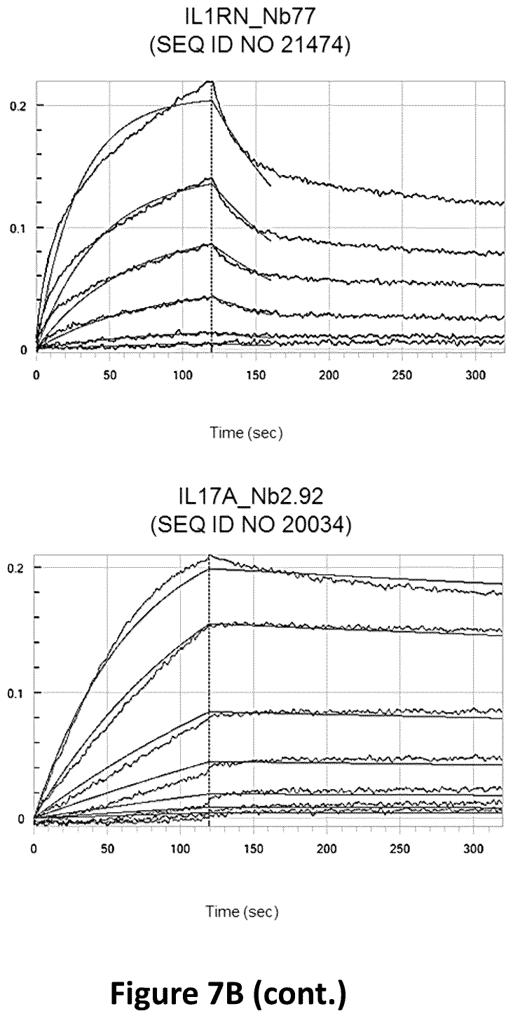

Octet RED96 System (ForteBio) was used to perform kinetic measurement of nanobody binding to antigens. For Anti-Penta-His Biosensors (HIS1K), his-tagged recombinant human proteins were first immobilized onto the biosensors, and recombinant nanobodies fused with human IgG1 were applied to detect binding. For Anti-Human IgG Fc Capture Biosensors (AHC), recombinant nanobodies fused with human IgG1 were first loaded onto the biosensors, and probes were dipped in serial dilutions of recombinant human antigens. FIG. 7 provides representative examples of BLI assay results that demonstrate specific and high-affinity binding of the nanobody:antigen pairs.

### REAP Assays

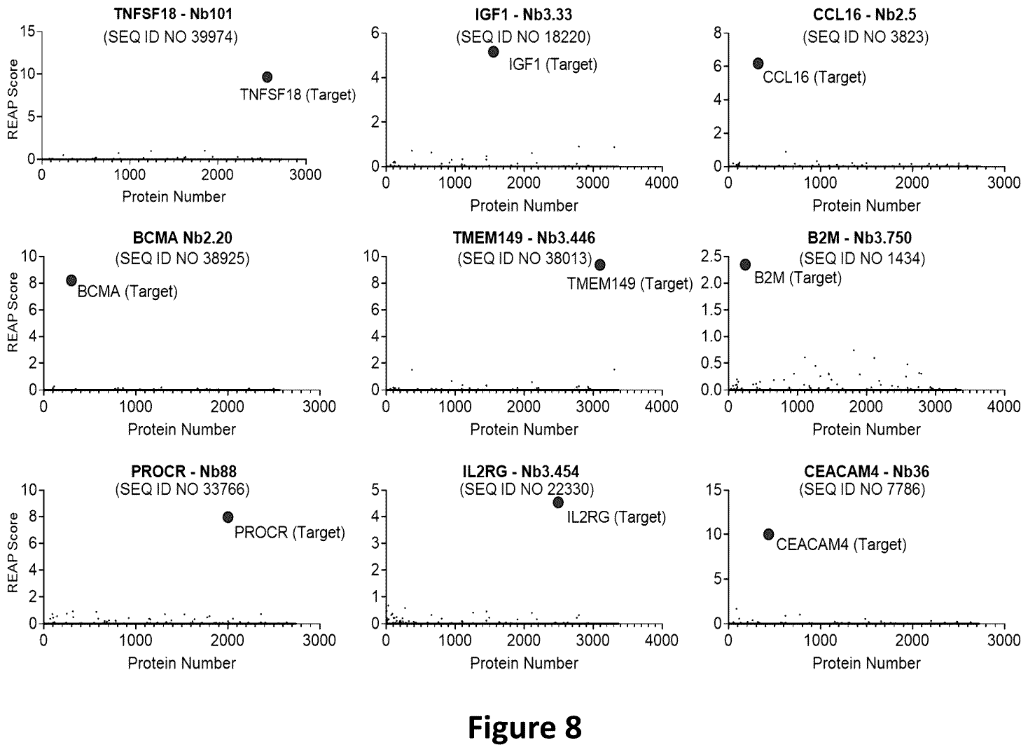

REAP (Rapid Extracellular Antigen Profiling) was used to profile the specificity of nanobodies. REAP was described in Wang, E., Dai, Y, Rosen, C., et al. bioRxiv, 2021 as a technology to profile interaction of an antibody against the human exoproteome. In brief, individual nanobodies were applied to a genetically barcoded, yeast-displayed human antigen library, and nanobody-bound yeast were magnetically enriched via Protein G coated beads. The selected yeast populations were subjected to next generation sequencing. The barcode information was used to calculate REAP Scores to describe the binding profiles of nanobodies to the human exoproteome. An interaction of a REAP Score above 2 is considered significant. FIG. 8 provides examples of REAP assay results for a select set of nanobodies. These results indicate that the recombinant nanobodies bind their antigen targets predicted via HAPPY with a high degree of specificity.

Table 1 provides data on a subset of the identified nanobodies that were further validated.

TABLE 1
Validated Nanobodies

| | | | | BLI AHC | BLI | REAP | |
|---|---|---|---|---|---|---|---|
| | | | ELISA | Sensor | HIS1K | Binding | REAP |
| SEQ ID | | | EC50 | KD | Sensor | to | Off- |
| NO: | Nb Name | Target | (M) | (M) | Binding | Targets | Targets |
| 1434 | B2M_Nb3.750 | B2M | N.A. | N.A. | Yes | Yes | 0 |
| 3779 | CCL16_Nb2.1 | CCL16 | 8.82E−10 | 1.74E−08 | N.A. | Yes | 2 |
| 3823 | CCL16_Nb2.5 | CCL16 | 1.78E−09 | N.A. | N.A. | Yes | 0 |
| 5485 | CD302_Nb26 | CD302 | 8.50E−09 | N.A. | N.A. | Yes | >10 |
| 5525 | CD302_Nb28 | CD302 | 6.84E−10 | N.A. | N.A. | Yes | 9 |
| 5523 | CD302_Nb30 | CD302 | 2.21E−09 | N.A. | N.A. | Yes | >10 |
| 5506 | CD302_Nb31 | CD302 | 1.26E−09 | N.A. | N.A. | Yes | >10 |
| 5519 | CD302_Nb32 | CD302 | 4.90E−10 | N.A. | N.A. | Yes | >10 |
| 5484 | CD302_Nb33 | CD302 | 2.26E−09 | N.A. | N.A. | Yes | >10 |
| 5524 | CD302_Nb35 | CD302 | 6.54E−10 | N.A. | N.A. | Yes | >10 |
| 5830 | CD3D_Nb2.33 | CD3D | 8.22E−09 | N.A. | N.A. | Yes | 1 |
| 5831 | CD3D_Nb2.34 | CD3D | 5.75E−10 | N.A. | N.A. | Yes | 2 |
| 5833 | CD3D_Nb2.35 | CD3D | 1.72E−09 | N.A. | N.A. | Yes | 3 |
| 5832 | CD3D_Nb2.36 | CD3D | 2.96E−08 | N.A. | N.A. | Yes | 1 |
| 5922 | CD3E_Nb2.73 | CD3E | 7.35E−10 | N.A. | N.A. | Yes | >10 |
| 5921 | CD3E_Nb2.74 | CD3E | 1.20E−09 | N.A. | N.A. | Yes | 2 |
| 5923 | CD3E_Nb2.76 | CD3E | 1.61E−08 | N.A. | N.A. | Yes | >10 |
| 5920 | CD3E_Nb2.77 | CD3E | 6.70E−10 | N.A. | N.A. | Yes | 8 |
| 5918 | CD3E_Nb2.79 | CD3E | 5.79E−10 | N.A. | N.A. | Yes | 7 |
| 5919 | CD3E_Nb2.80 | CD3E | 1.79E−08 | N.A. | N.A. | Yes | >10 |
| 7786 | CEACAM4_Nb36 | CEACAM4 | 1.56E−08 | N.A. | N.A. | Yes | 0 |
| 7787 | CEACAM4_Nb40 | CEACAM4 | 1.45E−08 | N.A. | N.A. | Yes | 5 |
| 7788 | CEACAM4_Nb41 | CEACAM4 | 3.27E−08 | N.A. | N.A. | Yes | 7 |
| 18220 | IGF1_Nb3.33 | IGF1 | N.A. | N.A. | N.A. | Yes | 0 |
| 19216 | IL10RA_Nb72 | IL10RA | 1.69E−08 | N.A. | N.A. | Yes | 8 |
| 19836 | IL13_Nb2.41 | IL13 | 1.02E−09 | N.A. | N.A. | Yes | 6 |
| 19837 | IL13_Nb2.42 | IL13 | 3.84E−08 | N.A. | N.A. | Yes | 0 |
| 20033 | IL17A_Nb2.90 | IL17A | 2.49E−10 | 9.77E−10 | N.A. | Yes | 3 |
| 20034 | IL17A_Nb2.92 | IL17A | 1.79E−10 | 6.56E−10 | N.A. | Yes | 0 |
| 21477 | IL1RN_Nb75 | IL1RN | 6.90E−09 | N.A. | N.A. | Yes | 0 |
| 21474 | IL1RN_Nb77 | IL1RN | 1.68E−10 | 8.52E−07 | N.A. | Yes | 5 |
| 21842 | IL21_Nb2.28 | IL21 | 3.14E−09 | N.A. | N.A. | Yes | 5 |
| 21838 | IL21 Nb2.29 | IL21 | 3.73E−09 | N.A. | N.A. | Yes | 7 |
| 21841 | IL21_Nb2.30 | IL21 | 7.16E−09 | N.A. | N.A. | Yes | 1 |
| 22330 | IL2RG_Nb3.454 | IL2RG | N.A. | N.A. | Yes | Yes | 0 |
| 24801 | LAIR2_Nb2.51 | LAIR2 | 3.79E−10 | N.A. | N.A. | Yes | 0 |
| 33801 | PROCR_Nb83 | PROCR | 1.27E−09 | N.A. | N.A. | Yes | 0 |
| 33767 | PROCR_Nb85 | PROCR | 3.52E−10 | N.A. | N.A. | Yes | 0 |
| 33867 | PROCR_Nb87 | PROCR | 1.04E−11 | 9.21E−08 | N.A. | Yes | 3 |
| 33766 | PROCR_Nb88 | PROCR | 2.87E−10 | 6.29E−08 | N.A. | Yes | 0 |
| 33765 | PROCR_Nb89 | PROCR | 5.65E−10 | N.A. | N.A. | Yes | 0 |
| 33768 | PROCR_Nb90 | PROCR | 1.74E−09 | N.A. | N.A. | Yes | 1 |
| 37689 | TIGIT_Nb2.81 | TIGIT | 2.98E−08 | N.A. | N.A. | Yes | 0 |
| 37690 | TIGIT_Nb2.82 | TIGIT | 1.28E−08 | N.A. | N.A. | Yes | 0 |
| 37688 | TIGIT_Nb2.84 | TIGIT | 4.83E−07 | N.A. | N.A. | Yes | 0 |
| 38013 | TMEM149_Nb3.446 | TMEM149 | N.A. | N.A. | N.D. | Yes | 0 |
| 38006 | TMEM149_Nb3.530 | TMEM149 | N.A. | N.A. | N.D. | Yes | 0 |
| 38406 | TNF_Nb105 | TNF | 5.57E−09 | N.A. | N.A. | Yes | 2 |
| 38381 | TNF_Nb109 | TNF | 2.38E−08 | N.A. | N.A. | Yes | 7 |
| 38949 | TNFRSF17_Nb2.18 | TNFRSF17 | 2.82E−09 | N.A. | N.A. | Yes | 1 |
| 38841 | TNFRSF17_Nb2.19 | TNFRSF17 | 2.84E−09 | N.A. | N.A. | Yes | 3 |
| 38925 | TNFRSF17_Nb2.20 | TNFRSF17 | 1.02E−09 | N.A. | N.A. | Yes | 0 |
| 38950 | TNFRSF17_Nb2.21 | TNFRSF17 | 4.75E−09 | N.A. | N.A. | Yes | 5 |
| 38951 | TNFRSF17_Nb2.23 | TNFRSF17 | 7.57E−09 | N.A. | N.A. | Yes | 2 |
| 39974 | TNFSF18_Nb101 | TNFSF18 | 2.26E−08 | N.A. | N.A. | Yes | 0 |
| 39973 | TNFSF18_Nb93 | TNFSF18 | 1.59E−09 | N.A. | N.A. | Yes | 0 |
| 40065 | TNFSF18_Nb94 | TNFSF18 | 7.18E−08 | N.A. | N.A. | Yes | 1 |
| 39972 | TNFSF18_Nb96 | TNFSF18 | 4.07E−09 | N.A. | N.A. | Yes | 6 |
| 39971 | TNFSF18_Nb98 | TNFSF18 | 6.05E−09 | N.A. | N.A. | Yes | 4 |

### Example 5: Prediction of High-Confidence Nanobodies Using Next Generation Sequencing Data

As part of the HAPPY selection workflow, nanobody pools before and after each round of the selection were subjected to next generation sequencing, which provided information on the binding behavior of each unique nanobody during the selection pressure. For instance, nanobodies that increased in abundance after the selection process indicated binding to the antigen. For another instance, nanobodies with high read counts for multiple antigenic targets indicate poly-specificity or poly-reactivity. Utilizing the next generation sequencing data, a computational pipeline was developed to select the highest-confidence nanobodies from the 24 billion nanobody variants in the library. Nanobodies predicted to have favorable outcomes in terms of both affinity and specificity to the targets were identified by applying the following steps:
1. Nanobody sequences with an internal stop codon were removed.
2. Next generation sequencing data were de-noised by filtering out sequences with less than 5 read counts. This cutoff was chosen to maximize the reproducibility of sequencing results between replicate samples.
3. Only nanobodies that were consistently detected before and after the selection procedure were considered.
4. Nanobodies with read counts greater than 200 were selected. This criterion puts the nanobody read count in the 97^{th} percentile of total read count, increasing the confidence in the affinity of the nanobody to a given antigen.
5. Nanobodies that were detected against less than 5 antigens were chosen. This step limits the poly-specificity of nanobodies with respect to antigens.

Using this computational pipeline, 42843 nanobodies against 1068 antigens were identified and included in the Sequence Listing. For each of the nanobodies in the sequences listing, the CDR1 sequence is defined as the region flanked by GSLRLSCAAS (SEQ ID NO:42891) GWYRQAPGKE (SEQ ID NO:42892); the CDR2 sequence is defined as the region flanked by WYRQAPGKER (SEQ ID NO:42893) . . . ADSVKGRFTI (SEQ ID NO:42894); and CDR3 is defined as the region flanked by KPEDTAVYYC (SEQ ID NO:42895) . . . WGQGTQVTVS (SEQ ID NO:42896). For example, the CDR1, CDR2, and CDR3 of SEQ ID NO:1 are aa26-34, aa46-59, and aa96-111, respectively.

Lengthy table referenced here
US20230416942A1-20231228-T00001
Please refer to the end of the specification for access instructions.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

LENGTHY TABLES
The patent application contains a lengthy table section. A copy of the table is available in electronic form from the USPTO web site (https://seqdata.uspto.gov/?pageRequest=docDetail&DocID=US20230416942A1). An electronic copy of the table will also be available from the USPTO upon request and payment of the fee set forth in 37 CFR 1.19(b)(3).

What is claimed is:

1. An isolated antibody or antibody fragment that specifically binds to a target protein or peptide, wherein the target protein or peptide is selected from the group consisting of a secreted protein or peptide and an extracellular protein or peptide.

2. The antibody or antibody fragment of claim 1, wherein the antibody or antibody fragment is selected from the group consisting of a monoclonal antibody, a polyclonal antibody, a single chain antibody, an immunoconjugate, a glycoengineered antibody, and a bispecific antibody or other multispecific antibody.

3. The antibody or antibody fragment of claim 2, wherein the antibody is a single chain antibody, and further wherein the single chain antibody is a nanobody.

4. The antibody or antibody fragment of claim 3, wherein the nanobody has a predicted binding score of at least 0.5, and further wherein the predicted binding score is determined using a machine learning algorithm wherein the machine learning algorithm comprises a multi-class classifier based on a logistic regression model.

5. The antibody or antibody fragment of claim 1, comprising an antibody or binding portion thereof that specifically binds to the secreted or extracellular protein or peptide with an affinity of at least 10⁻⁶ M.

6. The antibody or antibody fragment of claim 2, wherein the antibody or antibody fragment comprises a therapeutic agent or a detection moiety.

7. The antibody or antibody fragment of claim 2, wherein the antibody is selected from the group consisting of a humanized antibody, a chimeric antibody, a fully human antibody, an antibody mimetic.

8. The antibody or antibody fragment of claim 3, wherein the secreted or extracellular protein or peptide is selected from the group consisting of a target provided in Table 2.

9. The antibody or antibody fragment of claim 8, wherein the extracellular protein or peptide is selected from the group consisting of CCL16, CD302, CD3D, CD3E, CEACAM4, ILlORA, IL13, IL17A, IL1RN, IL21, PROCR, TIGIT, TNF, TNFRSF17, and TNFSF18.

10. The antibody or antibody fragment of claim 8, wherein the antibody comprises at least one selected from the group consisting of: a) the heavy chain CDR1 sequence selected from the group consisting of a CDR1 of SEQ ID NO:1-SEQ ID NO:42890 comprising a sequence between the flanking sequence of SEQ ID NO:42891 and SEQ ID NO:42892; b) the heavy chain CDR2 sequence selected from the group consisting of a CDR2 of SEQ ID NO:1-SEQ ID NO:42890 comprising a sequence between the flanking sequence of SEQ ID NO:42893 and SEQ ID NO:42894; and c) the heavy chain CDR3 sequence selected from the group consisting of a CDR3 of SEQ ID NO:1-SEQ ID NO:42890 comprising a sequence between the flanking sequence of SEQ ID NO:42895 and SEQ ID NO:42896.

11. The antibody or antibody fragment of claim 8, wherein the antibody comprises each of: a) the heavy chain CDR1 sequence selected from the group consisting of a CDR1 of SEQ ID NO:1-SEQ ID NO:42890 comprising a sequence between the flanking sequence of SEQ ID NO:42891 and SEQ ID NO:42892; b) the heavy chain CDR2 sequence selected from the group consisting of a CDR2 of SEQ ID NO:1-SEQ ID NO:42890 comprising a sequence between the flanking sequence of SEQ ID NO:42893 and SEQ ID NO:42894; and c) the heavy chain CDR3 sequence selected from the group consisting of a CDR3 of SEQ ID NO:1-SEQ ID NO:42890 comprising a sequence between the flanking sequence of SEQ ID NO:42895 and SEQ ID NO:42896, wherein each of the CDR1, CDR2 and CDR3 sequence are from the same SEQ ID NO.

12. The antibody or antibody fragment of claim 8, wherein the antibody comprises an amino acid sequence as set forth in any one of SEQ ID NO:1-SEQ ID NO:42890.

13. A composition comprising an antibody or antibody fragment of any one of claims 1-12.

14. A nucleic acid molecule comprising a nucleotide sequence encoding an antibody or antibody fragment of any one of claims 1-12.

15. A composition comprising a nucleic acid molecule of claim 14.

16. An expression vector comprising the nucleic acid molecule of claim 14.

17. A host cell comprising the nucleic acid molecule of claim 14.

18. A method of treating or preventing a disease or disorder in a subject in need thereof, the method comprising the step of administering the antibody or antibody fragment of any one of claims 1-12, the nucleic acid molecule of claim 14, or the composition of claim 13 or 15 to the subject.

19. A method of detecting a target secreted or extracellular protein or peptide in a sample, the method comprising:
a) contacting the sample with the antibody or antibody fragment of any one of claims 1-12, or the composition of claim 13, and
b) detecting binding of the antibody or antibody fragment of any one of claims 1-12, or the composition of claim 13 to the target secreted or extracellular protein or peptide.

20. A method of diagnosing a disease or disorder in a subject in need thereof, the method comprising
a. Contacting a biological sample of the subject with the antibody or antibody fragment of any one of claims 1-12, or the composition of claim 13,
b. determining the level of the target secreted or extracellular protein or peptide in the biological sample of the subject,
c. comparing the level of the target secreted or extracellular protein or peptide in the biological sample of the subject with a comparator control, and
d. diagnosing the subject with a disease or disorder associated with a differential level of the target secreted or extracellular protein or peptide when the level of the target secreted or extracellular protein or peptide in the biological sample of the subject of subject is significantly different when compared with the level of the target secreted or extracellular protein or peptide in the comparator control.

21. The method of claim 20, comprising the further step of administering a treatment to the subject that was diagnosed as having a disease or disorder.

22. The method of claim 20, wherein the comparator control is at least one selected from the group consisting of: a positive control, a negative control, a historical control, a historical norm, or the level of a reference molecule in the biological sample.

23. A method of selecting an antibody or antibody fragment of claim 1, the method comprising the steps of:
a) contacting a support comprising a display library of target secreted and extracellular proteins or peptides with a phage display library of candidate antibodies or antibody fragments, such that a complex is formed between the candidate antibody and the target secreted and extracellular proteins or peptide;
b) cleaving the complex from the support using a protease;
c) isolating the nucleic acid molecule encoding the antibody or antibody fragment; and
d) sequencing the nucleic acid molecule encoding the antibody or antibody fragment.

24. The method of claim 23, wherein the method further comprises predicting the binding score of the antibody or antibody fragment using a machine learning algorithm, wherein the machine learning algorithm comprises a multi-class classifier based on a logistic regression model.
